(19) United States
(12) Patent Application Publication
(10) Pub. No.: US 2026/0315627 A1
(43) Pub. Date: Oct. 15, 2026

(54) METHOD

(71) Applicant: NEUROCHASE INNOVATIONS LTD, Bristol (GB)

(72) Inventors: Neil Barua, Bristol (GB); Steven Streatfield Gill, Bristol (GB)

(21) Appl. No.: 19/483,419

(22) PCT Filed: May 10, 2024
(86) PCT No.: PCT/GB2024/051221

§ 371 Date: Nov. 11, 2025

(30) Foreign Application Priority Data
May 11, 2023 (GB) 2306952.9

(51) Int. Cl. A61K 48/00; C12N 15/86

(52) U.S. Cl. CPC A61K 48/0075; A61K 48/0033; C12N 15/86; C12N 2750/14143

(57) ABSTRACT

The present invention provides a gene or RNA therapy vector for use in the treatment of a non-malignant CNS disorder, wherein the gene or RNA therapy vector is to be infused into the pons via convection enhanced delivery (CED). Methods for treating a non-malignant CNS disorder, comprising infusing a gene or RNA therapy vector into the pons of a subject in need thereof via (CED) and methods of distributing a gene therapy throughout the nervous system, wherein a viral vector that is axonally transported anterogradely and retrogradely is infused into the pons and middle cerebellar peduncle of a subject via a cannula that is inserted through the cerebellum, the middle cerebellar peduncle and into the pons are also provided.

### FIELD OF INVENTION

The present invention relates to gene or RNA therapy vectors for use in the treatment of a non-malignant CNS disorder, wherein the gene or RNA therapy vectors are administered by convection enhanced delivery (CED).

### BACKGROUND TO THE INVENTION

Gene and RNA therapies are being developed for a wide range of neurological disorders affecting the motor and sensory systems. However, delivery of these therapies to clinically-relevant targets in the motor and sensory systems is highly challenging due to the complexity and distribution of their respective subsystems throughout the central nervous system (CNS).

Control of motor function (movement) requires complex and multifaceted co-ordination of neural signals from interconnected areas of the CNS including the cerebral cortex (primary, pre- and supplementary motor areas), motor thalamus, basal ganglia, brainstem, cerebellum, descending (corticospinal) tracts of the spinal cord and ultimately the peripheral nervous system. A process of continuous feedback, modification and fine-tuning also occurs with inputs from the sensory components of the CNS including peripheral nerves, dorsal root ganglia, ascending (spinothalamic) tracts of the spinal cord, sensory thalamus and sensory cortex. Reciprocal connections between the motor and sensory pathways also exist at all levels. Input from the visual and auditory systems are also required to modify and adjust motor control.

Motor and sensory functions are continuously modulated by the limbic system that comprises structures that include the temporal lobes, amygdala and hippocampus which have important roles in emotional and social processing, motivation, memory and learning.

Sensory systems are also paramount for control of movement. The influence of the sensory systems is complex and highly integrated, requiring co-ordination of inputs from the visual, auditory, vestibular and somatosensory components of the nervous system. One example of a neural circuit fundamental for motor control subserves proprioception (the subconscious understanding of limb position in space). Components of this circuit include muscle spindles, ascending tracts within Clarke's column, the dorsal columns, the nucleus gracilis and cuneatus, the medial lemniscus, the sensory thalamus and the sensory cortex.

A wide range of neurodegenerative conditions affect multiple components of the neural systems underpinning motor control and motor-sensory integration. Gene and RNA therapies have the potential to reverse or slow the progression of many neurological diseases if their vectors can be delivered in effective concentrations to affected CNS tissue. For many neurological diseases pathological change affects widely distributed motor regions of the brain, brainstem, cerebellum and spinal cord, as well as sensory components of the CNS including sensory nuclei and tracts. For example, generalised sensory abnormalities are associated with Amyotrophic Lateral Sclerosis (ALS), a neurodegenerative disorder considered to be primarily a disease of motor neurons.

An important limitation to the treatment of neurological disease is the presence of the blood-brain-barrier (BBB) which prevents many systemically administered therapeutic agents from reaching effective concentrations in the central nervous system (CNS), including viral vector-mediated gene therapies. A number of drug delivery strategies have been used to administer gene therapies for the treatment of neurological disorders, particularly Adeno-associated virus (AAV)-based vectors, characterised by degeneration of motor and/or sensory components of the CNS. Systemic, intra-CSF (intrathecal and cisterna magna) and intraparechymal delivery has been investigated. However, all of these strategies have limitations and risks which undermine the ultimate aim of safely, accurately and effectively achieving therapeutic transgene expression within the interconnected neuroanatomical targets subserving motor-sensory integration. These strategies and their deficiencies include:

### Systemic (Intravenous) Delivery:

E.g. Zolgenesma for the treatment of spinal muscular atrophy.
High dose and volume of vector required at high cost.
Poor CNS penetration limits efficacy (less than 1% of the dose of most systemically administered drugs reach the brain (Kimura & Harashima, 2020)).
Non-specific targeting of the CNS.
Associated with systemic toxicity including hepatotoxicity.
Circulating neutralising antibodies can negate effect.

### Intra-CSF Delivery

### Intrathecal Delivery

E.g. AAV9/JeT-GAN for giant axonal atrophy (Bailey et al., 2018).
High dose and volume of vector is required at high cost.
CNS penetration is concentration dependent resulting in poor and variable CNS penetration which is not targeted.
High local concentrations can lead to inflammation of and damage to the dorsal root ganglia and sensory tracts.
There is rapid absorption into the systemic circulation.
Effective dosing of motor cortex and sub cortical motor nuclei has not been demonstrated.

### Intra-Cisterna Magna (ICM) Delivery

E.g. AXO-AAV-GM2 for Tay-Sachs or Sandhoff Disease (https://clinicaltrials.gov/ct2/show/NCT04669535).
Limitations of vector distribution are similar to intrathecal delivery but improved although patchy cortical distribution has been demonstrated.
ICM injection is now considered a very rare technique.
Known procedure risks include subarachnoid haemorrhage, brainstem injury, compromise of vertebral artery blood flow, death.

### Subpial Spinal Cord Delivery

E.g. AAV9-shRNA-SOD1 for ALS (Bravo-Hernandez et al., 2020).
The subpial space is only a potential space in the human spinal cord and surgically challenging to access.
There is a high-risk of neural and vascular damage when targeting the subpial space.
Damage to the spinal cord carries a significant risk of permanent neurological injury including paralysis.
CNS penetration is concentration gradient dependent and variable.
High risk of dorsal root ganglia toxicity.
Effective dosing of motor cortex and sub cortical motor nuclei has not been demonstrated.

### Convection Enhanced Delivery (CED) to the Brain Parenchyma

### Intra-Thalamic Delivery

E.g. Intrathalamic CED of AAV2-GDNF (Kells et al., 2009).
The motor nuclei of the thalamus are the Ventral Anterior (VA) and Ventral Lateral (VL) nuclei. The VA nucleus receives afferents from the basal ganglia and sends efferents to the premotor cortex. The VL nucleus receives afferents from the basal ganglia and the dentate nucleus and sends efferents to the premotor and primary motor nucleus. There are no direct motor axonal connections with the spinal cord or cranial nerve motor nuclei and so the therapeutic benefit of targeting the thalamus alone for motor disorders may be limited.
The combined volumes of the motor nuclei of thalamus, the Ventral Anterior (VA) and Ventral Lateral (VL) nuclei, are approximately 1,400 mm³ per hemisphere. Typically, using the method of CED, the volume of distribution of an infusate in grey matter is ~4 times the volume infused so that 1,400 mm³ will be covered with 350 μL. This relatively small volume limits the dose of vector that can be safely delivered and reduces the likelihood of achieving therapeutic levels in the widely distributed motor system.
Damage to the thalamus is associated with risks such as thalamic pain syndromes.

### Combined Intrathalamic and Brainstem Delivery:

E.g. CED of AAV2-hASM-HA for Niemann-Pick disease Type A (Salegio et al., 2010).
AAV2 delivered to the brainstem in Non-Human Primates via a transfrontal trajectory in combination with delivery to the thalamus increased vector delivery to the cortex when compared with delivery to the thalamus alone. However, there was no report of transgene expression in spinal cord or cerebellum.

### Motor Cortex Delivery:

E.g. CED of optogenetic adeno-associated viral vector to the cortex of rhesus macaques (Khateeb et al., 2019)
whilst transgene expression in cortex was demonstrated (as well as in secondary motor neurons in comparable rat studies e.g. Nieuwenhuis et al., 2021) this technique has a number of disadvantages
coverage of only small volumes of motor cortex is possible
the translational potential of this method is limited due to the convoluted and complex shape of the human motor cortex
the thickness of the human motor cortex (only 1-4.5 mm) makes accurate targeting extremely challenging
these studies did not identify transgene expression in cerebellar circuits
similarly, white matter CED of AAV (Barua et al, 2013) resulted in transgene expression within the motor cortex but not in distal components of the motor pathways or cerebellar circuits.

Importantly, none of these strategies can effectively target the cerebellar circuits which are of fundamental importance for motor control, namely the dentatorubrothalamic, olivo-cerebellar or cerebrocerebellar circuits. Hence, there are still needs for improved methods for delivering required concentrations of drugs or other therapeutic agents to the regions of the CNS where they are most needed, particularly neurological conditions characterised by widespread neurodegeneration in structures required for effective motor-sensory integration.

In 2022 Salegio et al. reported the expression of AAV5-GFP following a single CED infusion into the cerebellum through a posterior, trans-tentorial trajectory in NHPs. GFP expression was identified in the cerebellum, DCN and related cerebellar tracts, but only inconsistently identified expression in the spinal cord (1 out of 4 test subjects). Of note, no transgene expression was identified in the motor cortex in any animal.

The connections between the motor systems, their anatomical locations and the fundamentally important influence of the cerebellar circuits means that the pons and the middle cerebellar peduncle are uniquely located as a site for targeted delivery of therapeutics. Specifically, at the axial level of the middle cerebellar peduncle within the pons lie the corticospinal tracts connecting the primary motor cortex to the thalamus and spinal cord; corticobulbar tracts from the premotor cortex to the pontine nuclei; the dentate nucleus connecting to cerebellum, motor thalamus, and inferior olivary nucleus. Furthermore, posterior to the corticospinal tracts lies the medial lemniscus carrying second-order neurons of the dorsal columns (a component of the sensory system transmitting proprioception, vibration sense, fine touch and 2-point discrimination). In close proximity are the superior cerebellar peduncles which are connected to the dentate nucleus and thalamus, red nuclei, and the pons is also the origin of the motor trigeminal nerve.

### SUMMARY OF THE INVENTION

To address the deficiencies of the above drug delivery methods and routes of administration, the present inventors investigated convection-enhanced delivery of viral vectors to the pons, targeting the ascending, descending, and transverse white matter tracts in order to exploit antero- and retro-grade transport and facilitate transgene expression in distal target structures through a trans cerebellar, middle cerebellar peduncle (MCP) trajectory. Advantageously, this trajectory may be used to deliver a gene therapy to the cerebellum and deep cerebellar nuclei (DCN) in addition to delivery to the pons.

The inventors hypothesised that due to the unique and extensive connectivity of nuclei and tracts within the pons and cerebellum, the trans MCP infusion of a retrogradely and anterogradely transported viral vector into this relatively small CNS volume, has the potential to transduce all the major neuronal circuits participating in sensory-motor processing. This includes the pyramidal and extrapyrarnidal motor pathways as well as the limbic, somatosensory, visual and auditory circuits that modulate motor activity.

The present inventors delivered viral vectors to the pons and cerebellum in sheep using the method of convection enhanced delivery (CED) through a canula inserted with trajectory through the cerebellum and MCP to the pons. The vectors AAV5 and AAV9 that are known to be axonally transported both anterogradely and retrogradely were carrying a transgene for the fluorescent marker protein, mCherry which is a non-secretory protein. One month after the infusion, postmortem analysis of the sheep's central and peripheral nervous system demonstrated extensive transgene expression in the frontal, parietal temporal and occipital cortices, the thalamus and basal ganglia, the hippocampus and amygdala, the brainstem, the spinal cord, the cranial nerves, including the optic tract, the peripheral nerves, including the sciatic nerve and muscle. This novel route of administration of gene therapies to the CNS overcomes many of the limitations of alternative routes.

The extensive CNS and peripheral nerve distribution covering the key neural pathways for sensory-motor processing that may be achieved by delivering antero and retrogradely transported viral vectors to the pons and cerebellum via a trans MCP trajectory is due to their widely distributed connectivity as follows:

### Connectivity of the Pons

The pons is the middle portion of the brainstem between the midbrain (rostral) and the medulla oblongata (caudal). It contains cranial nerve and other nuclei as well as ascending, descending and transverse tracts with connectivity as follows:

Cranial nerve nuclei: Sensory nuclei of trigeminal nerve, motor nucleus of trigeminal nerve; nucleus of the abducens nerve; nucleus of facial nerve; superior salivatory (salivary) nucleus; inferior salivatory (salivary) nucleus; vestibular nuclei; cochlear nuclei; solitary nucleus.
The motor nucleus of trigeminal nerve and facial nerve send efferents to the muscles of mastication and of facial expression respectively and receive corticobulbar afferents from the motor cortex.
The vestibular nuclei receive input on position, movement and balance from semicircular canals via the vestibular branch of vestibulocochlear nerve and other areas, including the spinal cord, contralateral vestibular nuclei and the reticular formation. In turn, these nuclei relay the received information to areas of the brainstem such as the reticular formation, oculomotor nuclei, thalamus, and cerebellum.
The cochlea nuclei relay auditory information through the lateral lemniscus, inferior colliculus, to the medial geniculate nucleus and auditory cortex. Efferents from the inferior colliculus to the superior colliculus contribute to the tectospinal tract that is part of the extrapyramidal system involved in orienting the eyes and the head towards sounds as part of the auditory and visual reflex.
The solitary nucleus receives special sensory information of taste as well as general sensory input from the tongue, palate and pharynxvia the facial, glossopharyngeal and vagus nerves. Its efferents are to the sensory thalamus and cortex, and to the brainstem reticular formation.
ynxvia the facial, glossopharyngeal and vagus nerves. Its efferents are to the sensory thalamus and cortex, and to the brainstem reticular formation.

Other nuclei in the pons: pontine nuclei, nucleus of the lateral lemniscus, nuclei of the pontine reticular formation, pedunculopontine nucleus, latero-dorsal tegmental nucleus, locus coeruleus.
The pontine nuclei receive extensive afferents from the frontal, parietal occipital and temporal cortex and in turn project pontocerebellar fibres to the contralateral cerebellar cortex and DCN via the middle cerebellar peduncle, establishing the cortico-ponto-cerebellar pathway. The cerebellum plays a significant role in cognitive and sensory-motor processing. Approximately 70-80% of the fibres in the middle cerebellar peduncle are from pre-frontal, temporal, parietal and limbic lobes and involved in cognition and the remaining 20-30% from sensory motor areas involved in motor processing. Retrograde transport of a viral vector along corticopontine fibres from a pons infusion may thus provide the means to achieve widespread cortical transduction.
nition and the remaining 20-30% from sensory motor areas involved in motor processing. Retrograde transport of a viral vector along corticopontine fibres from a pons infusion may thus provide the means to achieve widespread cortical transduction.
tion and the remaining 20-30% from sensory motor areas involved in motor processing. Retrograde transport of a viral vector along corticopontine fibres from a pons infusion may thus provide the means to achieve widespread cortical transduction.
The nucleus of the lateral lemniscus receives fibres from the cochlear nuclei and the superior olivary complex and projects to the superior and inferior colliculi. It is involved in the coordination of auditory and visual responses.
Nuclei of the pontine reticular formation (PRF) receive afferents from the pre-frontal, pre-motor and the motor cortex, parietal, and temporal lobes. These cortical inputs provide information related to motor planning and execution, sensory processing, and cognitive functions. Various brainstem nuclei send projections to the PRF, including the superior colliculus, the raphe nuclei, the locus coeruleus, and the vestibular nuclei. These connections are involved in coordinating motor responses, regulating arousal and sleep-wake cycles, modulating pain perception, and integrating sensory information. Reciprocal connections with the thalamic intralaminar and ventromedial nuclei contribute to arousal regulation, attention, and sensory processing and reciprocal connections with the cerebellum contribute to coordinating motor responses, adjusting muscle tone, and regulating eye movements. The PRF receives input from spinoreticular and corticoreticular tracts. These pathways convey sensory information from the body and limbs, contributing to motor control, reflex modulation, and pain processing. It sends efferents to the anterior horn cells of the spinal cord where it modulates muscle tone, reflex responsiveness, the coordination of locomotion and facilitation of antigravity muscle activity as well as serving as a pathway through which higher brain regions can influence motor activity at the level of the spinal cord.
The pedunculopontine nucleus (PPN) receives afferents from the cerebral cortex, including the primary motor cortex (M1), supplementary motor area (SMA), prefrontal cortex, and sensory cortices. The basal ganglia, including the globus pallidus, substantia nigra pars compacta (SNc) and the subthalamic nucleus (STN). These connections are involved in the regulation of motor functions, particularly in the context of movement initiation, coordination, and modulation. It also receives cerebellar afferents and afferents from brainstern nuclei including the reticular formation and the vestibular nuclei. These connections contribute to the modulation of arousal, attention, and sensory processing, as well as the coordination of motor responses. The amygdala and hippocampus also send afferents to the PPN potentially influencing its role in emotional processing and motivated behaviours. The PPN sends efferents to the SNc, the striatum, the basal forebrain, the amygdala, the mesencephalic locomotor region, the pontine reticular formation, the locus coeruleus, the superior colliculus and the spinal cord locomotor pattern generators.
The locus coeruleus projects fibres to various regions of the central nervous system, including the cerebral cortex, diencephalon, limbic system, brainstem, cerebellum and spinal cord. It plays a role in the wake-sleep cycle, attention and stress response.

Transverse tracts: fibres of cochlear nuclei, transverse pontine (pontocerebellar) fibres.

Ascending tracts: medial lemniscus, lateral lemniscus, spinal lemniscus (spinothalamic tract), trigeminal lemniscus (trigeminothalamic tract).
The medial lemniscus is formed by fibres from the gracile and cuneate nuclei within the dorsal medulla. It transmits proprioception, vibration and fine touch sensations from the dorsal column of the spinal cord to the VPL thalamus, and ultimately to the primary somatosensory cortex. Retrograde transport of a vector from the gracile and cuneate nuclei would be to first order sensory neurons with nuclei in the spinal cord dorsal root ganglia.
The lateral lemniscus forms part of the auditory pathway and its fibers terminate in the inferior colliculus. It receives afferents from various nuclei, including the cochlear nuclei, superior olivary nucleus and the nucleus of the lateral lemniscus.
The spinal lemniscus (spinothalamic tract) is formed from the combination of the anterior and lateral spinothalamic tracts. It conveys coarse touch, pain and temperature information from the contralateral side of the body to the thalamus and to the primary somatosensory cortex. Retrograde transport will be to the first order sensory neurons with nuclei in the spinal cord dorsal root ganglia.

Descending tracts: corticospinal, corticobulbar, corticopontine, reticulospinal, rubrospinal, rubroreticular, vestibulospinal, and tectobulbospinal tracts.
The corticospinal tract arises predominantly from the primary motor cortex and descend via the basal pons to the medulla where the majority of fibres cross forming the pyramidal decussation, after which they continue on the contralateral side as the lateral corticospinal tract. The remaining fibres that do not cross over remain ipsilateral and continue caudally as the anterior corticospinal tract. The fibres of the corticospinal tract synapse with anterior horn motor neurons of the spinal cord and are responsible for voluntary movement of muscles of the trunk and limbs.
The corticobulbar tract arises from the motor cortex of the brain and descends to the motor nuclei of cranial nerves within the pons and medulla. It is responsible for voluntary motor control of the face, head and neck.
The corticopontine tract is a large group of fibres that originate from the frontal, parietal occipital and temporal cortex and terminate on neurons in the ipsilateral pontine nuclei as described above.
The pontine reticulospinal tract descends through the brainstem and spinal cord. It primarily terminates in the ventral horn of the spinal cord, where it synapses with lower motor neurons and interneurons. It is involved in facilitating and regulating voluntary and reflexive movements, particularly those related to posture and locomotion.
The rubrospinal tract descends from the red nucleus, crosses in the upper pons and descends into the spinal cord where it synapses with secondary motor neurons in the anterior horn. It is important for coordinating movements that require precision, such as reaching, grasping, and manipulating objects.
The rubroreticular tract serves to modulate and coordinate motor activity mediated by the reticular formation influencing the activity of reticulospinal neurons.
The vestibulospinal tract descends to the spinal cord through a lateral tract where it synapses on anterior horn cells to influence antigravity muscles of the limbs and trunk. It has a medial component that terminates primarily in the cervical region that influences neck muscles, particularly those involved in head and neck orientation and stabilization.
The tectobulbospinal tract originates in the superior colliculus, where it receives inputs from the retina and other visual pathways and sends projections to the brainstem, specifically to the reticular formation and cranial nerve nuclei, and to the spinal cord. This tract helps to coordinate the movements of the eyes, head, neck and body in response to visual stimuli, such as tracking moving objects or postural adjustments and balance in response to visual cues. Delivery of a retrogradely transported viral vector that transduces the tectobulbospinal tract and thus the superior colliculus may therefore transduce the optic tract, retina, and visual cortex.

Complex tracts: medial longitudinal fasciculus, central tegmental tract.
The medial longitudinal fasciculus is made up of both ascending and descending fibres that establish connections with the oculomotor, trochlear, abducens, Edinger-Westphal, vestibular, reticular and spinal accessory nuclei. Through these connections, and connections with the superior colliculus, the medial longitudinal fasciculus plays a role in coordinating conjugate eye movements and associated movements of the head and neck.
The central tegmental tract is a complex nerve bundle that spans the entire brainstem terminating in the inferior olivary nucleus. It contains descending fibres from the red nucleus that terminate in the ipsilateral inferior olivary nucleus, and the ascending gustatory fibres from the nucleus solitarius that terminate in the cortical taste area. The central tegmental tract also carries ascending and descending fibres of the brainstem reticular formation and serves as a pathway for connections between the midbrain to the cerebellum.

### Connectivity of the Middle Cerebellar Peduncle and Cerebellum

The infusion of a gene therapy vector along the axis of the middle cerebellar peduncle (MCP) will augment the transduction of ponto-cerebellar tracts from the contralateral pontine nuclei to the cerebellar cortex and Deep Cerebellar Nuclei and importantly will encompass the inferior cerebellar peduncle and the dentate nucleus. Due to the unique connectivity of the inferior cerebellar peduncle and the dentate nucleus, their inclusion provides the means transduce several key structures that are essential for effective sensory-motor and cognitive processing that would not be achievable with an infusion that was confined to the pons.

The inferior cerebellar peduncle (ICP) serves as a major pathway for conveying sensory information from various sources to the cerebellum. It contains several tracts, each with distinct connections and functions, which include:
1. Spinocerebellar Tracts:
   Dorsal Spinocerebellar Tract: Carries non-conscious proprioceptive information from the lower limbs and trunk to the cerebellum that is essential for normal motor function and originates from Clarke's nucleus in the spinal cord.
   Ventral Spinocerebellar Tract: Conveys proprioceptive information from the lower limbs and trunk to the cerebellum. Originates from neurons in the spinal cord and crosses over to the opposite side before ascending to the cerebellum.
2. Cuneocerebellar Tract:
   Conveys proprioceptive information from the upper limbs and cervical region to the cerebellum. Originates from Clarke's nucleus in the medulla oblongata.
3. Olivocerebellar Tracts:
   Climbing Fibers: Arise from neurons in the inferior olivary nucleus of the medulla oblongata. They form synapses directly onto Purkinje cells in the cerebellar cortex, providing powerful excitatory input. These fibres are crucial for motor learning and coordination.
4. Vestibulocerebellar Tracts:
   Carry vestibular input from the vestibular nuclei in the brainstem to the cerebellum. These fibres convey information about balance, spatial orientation, and head position.

The sensory information carried by these tracts is essential for proprioception, balance, and coordination, which are key functions of the cerebellum. Upon entering the cerebellum, the fibres from these tracts synapse with neurons in the cerebellar cortex and deep cerebellar nuclei, contributing to the integration of sensory and motor signals necessary for motor control and coordination. The cerebellum then processes this information and sends output to various motor centres in the brainstem and cerebral cortex, modulating motor commands and contributing to the refinement of movement patterns.

The Dentate nucleus receives the majority of its afferents from the cerebellar cortex, but these are indirect. Other afferents are from the contralateral pontine nuclei and from the inferior olivary nucleus conveying excitatory input, contributing to motor learning and coordination. Efferents from the dentate pass through the superior cerebellar peduncle to the contralateral thalamus and specifically to the ventrolateral thalamus (VL) and the ventroanterior thalamus (VA) and thence to the prefrontal, premotor and motor cortex. The dentate also has efferents to the amygdala (limbic system) and high functional connectivity (multisynaptic) with occipital, parietal and frontal components of visual attention and spatial cognition. Other efferents are to the cerebellar cortex and the contralateral red nucleus and the reticular formation. The dentate connections are involved in integrating limbic, cognitive and associative motor and sensory information that contribute to cognition, attentional control, visual and sensory processing, motor planning, sequencing, and coordination. The extensive connectivity of the dentate nucleus makes it a highly effective target from which to distribute transgenes with anterogradely and retrogradely transported vectors to the greater part of the cortex and subcortical structures.

The larger and generally ovoid volume of the pons (approx. 48 000 mm³ in adults) also contributes to its utility as a target for gene therapy. It is substantially larger than, for example the motor nuclei of the thalamus (1,400 mm³). It is possible to safely infuse large volumes of therapeutic agent into the pons without adverse clinical effects (Szychot et al., 2021) which facilitates optimisation of the therapeutic dose range for a gene therapy.

Accordingly, in a first aspect the present invention provides a gene or RNA therapy vector for use in the treatment of a non-malignant CNS disorder, wherein the gene or RNA therapy vector is to be infused into the pons via convection enhanced delivery (CED). Methods for treating non-malignant CNS disorders comprising infusing a gene or RNA therapy vector into the pons via CED are also provided. Optionally, the gene or RNA therapy vector is also infused into the middle cerebellar peduncle.

The gene or RNA therapy vector is administered by convection enhanced delivery (CED). Convection enhanced delivery is well known in the art. It means the delivery of a pharmaceutical, or other composition, to the brain under a positive pressure gradient via a narrow catheter, usually having an inner diameter of less than 500 μm, more usually less than 250 μm. Administration of the gene or RNA therapy vector or other composition by CED leads to a greater volume of distribution than is typically achieved by intracerebral injection or infusion.

The gene or RNA therapy vector may be infused into the anterior pons. Preferably, the gene or RNA therapy vector is infused into the pons and the middle cerebellar peduncle. Preferably, the infusion is through at least one cannula placed via a trans-cerebellar posterior to anterior trajectory traversing laterally to the deep cerebellar nuclei. The inventors have demonstrated that using this approach can achieve infusate distribution encompassing the DCN without risking injury to these important structures, i.e., without the need to place a cannula into or directly through the DCN. In embodiments of the invention, the infusion may be via two bilaterally positioned catheters, each placed via a trans-cerebellar posterior to anterior trajectory traversing laterally to the deep cerebellar nuclei. This trajectory may be used in conjunction with one or more transfrontal trajectories targeting the pons from a superior to inferior route.

The cannula may be a reflux resistant cannula, such as a stepped cannula e.g., as described in Gill et al 2013 (incorporated herein by reference). Preferably, the infusion is performed using a stepped cannula comprising a guide tube and a fluid transfer tube, wherein the fluid transfer tube is inserted through the guide tube and into the tissue. The guide tube creates a step where the fluid transfer tube emerges. Further preferably, the fluid transfer tube comprises four or more reflux regions on its outer surface which are configured to allow flow of the infusate from the distal end of the fluid transfer tube towards the proximal end of the fluid transfer tube along the reflux regions when the distal end of the fluid transfer tube is inserted into the tissue. The combination of the step and reflux regions can provide a more uniform and consistent distribution of the infusate around the fluid transfer tube. Such cannulas are described in detail in GB 2307199.6 incorporated herein by reference.

The cannula step may be placed at the interface of the posterior deep cerebellar nuclei (DCN) and cerebellar white matter. Indeed, the present inventors have demonstrated that by using a stepped cannula, with the step placed at the posterior interface of the DCN and cerebellar white matter, it is possible to control reflux of the infusate. By refluxing infusate back to the step of the catheter it is possible to distribute infusate into the DCN without risking injury to these important structures and in addition encompass the inferior cerebellar peduncle which passes medial to the middle cerebellar peduncle in its more caudal aspect. The inferior cerebellar peduncle conveys proprioceptive information from the spinal cord, information from the inferior olivary nucleus pertinent to the timing and coordination of movements, and the vestibular nuclei conveying information about balance and spatial orientation to the cerebellum.

The gene or RNA therapy vector may comprise a viral or a non-viral vector.

Suitable viral vectors include adenoviruses, adeno-associated viruses (AAVs), lentiviruses, and herpes viruses. Preferably the viral vector is an AAV vector, such as AAV5 or AAV9. Alternatively, the viral vector may be an AAV hybrid such as AAV2/5.

The AAV serotypes most studied in the CNS have been serotypes 1, 2, 5, 6, 8, 9, and recombinant human (rh)10. The recombinant genome of a given serotype can be packaged into the capsid of another serotype (e.g., rAAV2/5 contains the AAV2 recombinant genome packaged in the capsid proteins encoded by the cap gene of AAV5). The AAV serotypes have been characterised in animal models and can be selected for targeted gene therapy based on their specific tissue tropisms, bio-distribution and transduction efficiency. Transfection of AAV serotypes 2 and 6 are neuronally restricted whereas serotypes 7, 9 and 5 transfect both neurons and astrocytes. Axonal transport of the AAV transgene capsid, either anterograde or retrograde, varies between serotypes and can be both target and dose dependent. AAV6 typically shows retrograde axonal transport, whereas AAVs 1, 2, 5, 8, and 9 have been shown to have either anterograde or retrograde axonal transport depending on the neuronal type transfected and/or the dose of vector delivered to the CNS tissue (Li et al., 2019).

Unilateral infusion of a gene or RNA therapy into the pons and middle cerebellar peduncle using a viral vector which is both antero- and retro-gradely trans may, due to the unique connectivity of the structures that would be encompassed in the infusion, distribute bilaterally in the cerebral and cerebellar cortex. This is because a unilateral infusion in pons that transduces the ipsilateral pontine nuclei will be distributed retrogradely to their widespread cortical afferents and anterogradely to the contralateral cerebellar cortex and DCN. The pontocerebellar fibres passing from the contralateral side along the ipsilateral middle cerebellar peduncle will also be transduced resulting in anterograde transport to the ipsilateral cerebellum and DCN and by retrograde transport to the contralateral cortex along their cortical afferents. Anterograde transport along efferent fibres from the cerebellar cortex and DCN have significant connectivity with the cerebral cortex and subcortical structures that will greatly enhance the distribution of therapeutic transgenes or RNA therapy. The pons provides a unique anatomical location where the pyramidal and extrapyramidal neural pathways converge facilitating their transduction in a relatively small CNS volume and their anterograde and retrograde transport to the cortex, subcortical structures and spinal cord. Similarly, ascending somatosensory pathways can be transduced in the pons and distributed to the cortex and spinal cord except for the dorsal spinocerebellar tract that passes through the inferior cerebellar peduncle to the cerebellum. However, as described above, the inferior cerebellar peduncle can be covered with an infusion along the middle cerebellar peduncle. Finally, the unique connectivity of the pons provides a location whereby transduction of special sensory pathways may also be achieved, as described above. When required, CNS distribution may be further augmented with bilateral infusions. This may also enable effective doses of vector to be administered to widely distributed CNS structures whilst reducing the concentration of the vector delivered through a single cannula, thereby reducing the chance of local toxicity.

The gene or RNA therapy vector may be administered at a concentration of about 1×10³ viral particles per ml to about 1×10²⁰ viral particles per ml, or about 1×10⁵ viral particles per ml to about 1×10¹⁵ viral particle per ml preferably about 1×10⁵ viral particles per ml to about 1×10¹⁵ viral particles per ml or about 1×10⁸ to about 1×10¹³ viral particles per ml. Flow rates for CED will typically range from 0.5 μl/minute to 20 μL/minute or 1 μL/minute to 20 μL/minute, preferably from 1 μL/minute to 10 μL/minute, more preferably from 1 μL/minute to 5 μL/minute. In embodiments of the invention the flow rate may be around 3 μL/minute.

The volume of infusion may be from about 50 μl to about 10 mL, or about 0.5 mL to about 5 mL, preferably from about 0.5 mL to about 3 mL, more preferably from about 0.5 mL to about 2 mL. In embodiments of the invention the volume of infusion may be about 1 mL or about 0.5 mL or about 0.3 mL. The infusion volumes described herein are usually volume per day.

The volume of infusion from a single cannula may be from about 0.5 mL to about 3 ml. One cannula can be inserted down each middle cerebellar peduncle so that a total infused volume with this strategy may be up to 6 ml. If two additional cannulas are inserted through the cerebral peduncles then these are preferably directed to the mid sagittal portion of the pons, with one cannula positioned more anteriorly to deliver to the basal pons and the other more posteriorly to deliver to the tegmental pons. With this latter arrangement four cannulas can be infused simultaneously with a total volume of infusion to the pons, MCPs and cerebellum of up to 10 ml. It is preferable to conduct the infusions in awake patients so that they can be monitored neurologically. The distribution of the infusate during the infusion can also be monitored with intermittent MRI scanning. Optionally, the gene therapy vector is co-infused with an MRI contrast agent such as Gadolinium-based contrast agents (GBCAs) such as gadobenate dimeglumine (MultiHance), gadobutrol (Gadavist), gadopentetate dimeglumine (Magnevist), gadoteridol (ProHance), and gadoterate meglumine (Dotarem). Alternative MRI contrast agents include iron oxide nanoparticles, ultrasmall superparamagnetic iron oxide particles or manganese-based contrast agents.

Suitable non-viral vectors include polymers, lipids, peptides, inorganic materials and hybrid systems. Polymer vectors can be non-biodegradable (e.g., polyethylenimine (PEI), Poly(vinylimidazole) (PVI) and PAMAM) or biodegradable (e.g., Chitosan, Poly(β-amino ester)s (PBAEs) and polylactide (PLA)). Examples of lipid vectors include conventional lipids, gemini surfactants, lipidoids and helper lipids. Peptide vectors include peptide nucleic acid conjugates, polypeptides and cell-penetrating peptides. Inorganic vectors include silica-based systems, such as mesoporous silica nanoparticles, gold nanoparticles, magnetic nanoparticles, carbon nanotubes, graphene, upconversion nanoparticles, and quantum dots. Examples of hybrid vector systems include inorganic-organic hybrid vectors, modified PEI vectors, inorganic-lipid and peptide-lipid vectors.

Gene or RNA therapy, as is well known, is the use of genetic material to modulate or add to genes in an individual's cells in order to treat disease. The genetic material to be introduced may be any appropriate genetic material, including DNA, RNA, small interfering RNA (siRNA), microRNA, short hairpin RNA (shRNA) or antisense oligonucleotide (AON) cassette. In embodiments of the invention the gene or RNA therapy may comprise CRISPR/Cas9. The gene or RNA therapy may be used to treat the disease in any known manner, such as gene replacement, gene knockdown, gene editing, pro-survival gene therapy and cell suicide therapy as well as expression of a neuroprotective and/or neurorestorative molecule such as a neurotrophin. In embodiments of the invention the gene or RNA therapy may be a monoclonal antibody-encoding nucleotide sequence, which may express full-length IgG, antibody-protein fusion products, such as immunoadhesins or bispecifics, or antibody fragments, such as antigen-binding fragments (Fab), single-chain variable fragments (scFv) or single domain antibodies.

Combinations of gene and/or RNA therapy vectors could be administered. For example, two different vectors each comprising the same genetic material or different genetic material may be administered sequentially or in combination (i.e., simultaneously). Sequential administration of two different vectors may be advantageous in the event that the first vector triggers or is at risk of triggering an immune response. Using a different vector for a further administration of the genetic material can avoid the immune response from being triggered. Additionally or alternatively, two or more different gene or RNA therapies may be administered using the same vector. The two or more gene or RNA therapies may be delivered sequentially or in combination (i.e., simultaneously).

The gene and/or RNA therapy vector described herein can be administered conjunction with one or more additional gene or RNA therapy vectors as described above and/or in conjunction with one or more other therapeutic agents, such as, histone deacetylase inhibitors or albumen. The additional gene and/or RNA therapy vectors or other therapeutic agents can be administered by systemic, intra-CSF, intraparenchymal or subpial spinal administration.

The present invention can be used for the treatment of neurological disorders characterised by pathological changes in the motor (pyramidal and extrapyramidal), cerebellar, sensory and limbic pathways as well as the cerebral cortex. Therapeutic indications are therefore considered according to these target structures, however, in most of the disorders listed, pathological changes are not confined to a single component of the nervous system, and in fact affect components of the cortical and subcortical motor, sensory and associative networks requiring widespread distribution of a therapeutic agent.

### Diseases Characterised by Degeneration of Predominantly Pyramidal Motor Pathways

Amyotrophic lateral sclerosis (ALS), formerly known as Lou Gehrig's disease, can affect the upper and/or lower motor neurons. It causes rapid loss of muscle control and eventual paralysis. Although degeneration of motor neurons is the hallmark of ALS, it is clear that loss of sensory pathways also affects patients and it may be associated with cognitive impairment.

Spinal muscular atrophy (SMA) refers to a group of hereditary diseases that affect lower motor neurons. The most common form is caused by a mutated or missing gene known as the survival motor neuron gene 1 (SMN1), which causes the neurons to deteriorate, producing muscle weakness and wasting.

Spinal muscular atrophy with respiratory distress type 1 (SMARDI) is a very rare form of SMA caused by mutations in the IGHMBP2 (immunoglobulin helicase μ-binding protein 2) gene. Symptoms appear during infancy, between ages 6 weeks and 6 months. Children with SMARDI suddenly may be unable to breathe due to diaphragm paralysis and may develop weakness in the muscles of their hands and feet.

Congenital SMA with arthrogryposis is a rare disorder that appears at birth. Symptoms include severe joint contractures, making babies unable to extend or flex the affected joints. In most children, both the arms and legs are affected. Other symptoms include scoliosis (curvature of the spine), chest deformity, respiratory problems, unusually small jaw, and drooping eyelids.

Progressive bulbar palsy (PBP), also known as progressive bulbar atrophy, is due to injury of the upper motor neurons in the brainstem or the lower motor neurons connected to the brainstem. The brainstem controls the muscles needed for swallowing, speaking, chewing, and other functions.

Primary lateral sclerosis (PLS) affects only the upper motor neurons, causing difficulty and slowness in the movements of the arms, legs, and face. Symptoms include weakness, muscle stiffness and spasticity, clumsiness, slowing of movement, and problems with balance and speech. The disorder often affects the legs first, followed by the torso, arms and hands, and, finally, the muscles used for swallowing, speaking, and chewing.

Progressive muscular atrophy (PMA) is an uncommon subtype of ALS marked by slow but progressive damage to the lower motor neurons. It affects men more often than women, and usually at symptoms begin later in life than typical ALS. People with PMA usually notice weakness in their hands or feet followed by spreads into other body regions. They may have weakness in the torso muscles and may have trouble breathing. Exposure to cold can worsen the person's symptoms. Other symptoms may include muscle wasting or shrinking, clumsy hand movements, twitches, and muscle cramps.

Kennedy's disease is an inherited lower motor neuron disorder that affects men. The onset of symptoms varies, but usually begins between the ages of 20 and 40. Kennedy's disease is also known as spinal and bulbar muscular atrophy (SBMA), bulbo-spinal muscular atrophy, or X-linked spinal and bulbar muscular atrophy. It is caused by mutations in the gene for the androgen (male sex hormone) receptor. Daughters of people with Kennedy's disease have a 50% chance of having a son affected with the disease.

Post-polio syndrome (PPS) usually occurs 15-40 years after a person has polio, an infectious viral disease. PPS is believed to be the result of deterioration of the motor neurons over many years that leads to loss of muscle strength and dysfunction. It is not contagious and only someone who has had polio can develop PPS. Not everyone who has had polio will develop PPS.

### Diseases Characterised by Degeneration of Predominantly Extrapyramidal Motor Pathways

Parkinson's disease and Parkinson's plus syndromes are characterised by muscle rigidity, rest tremor, and slowness of movement. In addition, autonomic and cognitive impairments commonly occur. widespread progressive pathological changes commencing in the lower brain stem and ascending to the midbrain, amygdala, thalamus and ultimately the cerebral cortex.

Huntington's disease is a hereditary disease affecting behaviour, cognition and movement resulting in uncontrolled rapid and jerky limb movements. Huntington's disease stems from a defect that consists of an expanded CAG repeat in the huntingtin gene (HTT).

Dystonia is a hyperkinetic movement disorder that is characterized by involuntary movement and the slowing of intentional movement. Though there are known causes of dystonia such as metabolic, vascular, and structural abnormalities, there are still patients with dystonia with no apparent cause. Dystonia can occur as a hyperkinetic disorder or as a side effect of hypokinetic disorders such as Parkinson's disease.

Hemiballismus is a hyperkinetic movement disorder that causes uncontrolled movement on one side of the body. It is generally caused by damage to the subthalamic nucleus (STN).

Tourette's syndrome is a disorder that is characterized by behavioural and motor tics, OCD and attention deficit hyperactivity disorder. It is thought to be caused by abnormalities of the motor, limbic and associative networks of the brain.

Sydenham's chorea is a disorder characterized by rapid, uncoordinated jerking movements primarily affecting the face, hands and feet. It is a result of an autoimmune response that occurs following infection by group A β-hemolytic streptococci (GABHS), resulting in damage of the basal ganglia.

Dyskinetic cerebral palsy is a subtype of cerebral palsy associated with damage to the basal ganglia during brain development due to bilirubin encephalopathy and hypoxic-ischemic brain injury. Symptoms include slow, uncontrolled movements of the extremities and trunk and small, rapid, random and repetitive, uncontrolled movements known as chorea. Involuntary movements often increase during periods of emotional stress or excitement and disappear when the patient is sleeping or distracted.

Athymormic syndrome is a rare psychopathological and neurological syndrome characterized by extreme passivity, apathy, blunted affect, and a profound generalized loss of self-motivation. The syndrome is believed to be due to damage to areas of the basal ganglia or frontal cortex, specifically the striatum and globus pallidus, responsible for motivation and executive functions.

Lesch-Nyhan syndrome is a rare X-linked recessive disorder caused by a deficiency of the enzyme hypoxanthine-guanine phosphoribosyltransferase (HGPRT), leading to uric acid build-up and a deficiency in dopamine production. Within the first few years of life, extrapyramidal involvement causes abnormal involuntary muscle contractions such as loss of motor control (dystonia), writhing motions (choreoathetosis) and arching of the spine (opisthotonus).

Wilson's disease is an autosomal recessive genetic disorder caused by a mutation in the copper-transport gene ATP7B, leading to excess copper build-up. Beurological symptoms include parkinsonism (most commonly cogwheel rigidity, bradykinesia or slowed movements and a lack of balance) with or without a typical hand tremor, masked facial expressions, slurred speech, ataxia or dystonia.

### Diseases Characterised by Degeneration of Predominantly the Cerebral Cortex

Alzheimer's disease is the most common form of dementia and is caused by degeneration of areas of cerebral cortex which control memory, language, reasoning and social behaviour.

Frontotemporal dementia is characterised by neuronal degeneration in primarily the frontal and temporal lobes of the brain.

Creutzfeld-Jacob disease is a rare degenerative brain disease which results in a rapidly progressive dementia.

### Diseases Characterised by Predominant Degeneration of the Cerebellar System

Friedreich's ataxia (FA) is an autosomal recessive genetic disease that has endocrine, cardiac and nervous system manifestations, and is caused by mutations on the FXN gene on chromosome 9. The FXN gene produces the protein frataxin, which is involved in iron-sulfur protein synthesis as part of normal mitochondrial homeostatic functioning. FA is, in the majority of cases, caused by a GAA trinucleotide repeat disorder on both alleles of the gene, which causes defective transcription of the FXN gene and, thus, reduces the levels of frataxin. Gene therapy targeted towards elevating levels of frataxin hold substantial potential for therapeutic benefit.

FA is the most common inherited ataxia (abnormalities of co-ordination, balance and speech). Neurological symptoms usually occur before the age of 20 and include difficulty walking, abnormalities of proprioception and sensation, weakness and muscle atrophy and spasticity. Loss of dorsal root ganglia, degeneration of peripheral nerves and the dorsal columns (sensory pathways) of the spinal cord results in the cerebellum being unable to co-ordinate movement. The dorsal spinocerebellar tracts, dentate nuclei, cortical motor neurons and corticospinal (motor) tracts also degenerate. The Purkinje cells of the cerebellum are also injured in FA. Purkinje cells are the only output neuron of the cerebellar cortex and their degeneration has significant adverse effects on cerebellar function. Visual impairment due to retinal degeneration may also occur in some individuals.

Spinocerebellar ataxia is characterised by cerebellar atrophy causing ataxia; loss of motor neurons, resulting in motor impairment; atrophy of the pons and medulla; and atrophy of the basal ganglia, and therefore represents another potential therapeutic target for pontine delivery.

### Diseases Characterised by Disorders of the Limbic System

Epilepsy is a brain disorder that causes recurring, unprovoked seizures.

Limbic encephalitis is a paraneoplastic phenomenon, results from antibody-mediated limbic system inflammation. As its clinical hallmark, over several days to several weeks, individuals develop pronounced memory impairment (amnesia) often accompanied by irritability and behavioral disturbances.

Dementias are a group of diseases characterised by cognitive decline interfering with activities of daily life.

Anxiety disorders are a group of mental health conditions including generalized anxiety disorder, panic disorder with or without agoraphobia, specific phobias, agoraphobia, social anxiety disorder, separation anxiety disorder and selective mutism.

Schizophrenia is a serious mental disorder in which people interpret reality abnormally. Schizophrenia can be characterised by hallucinations, delusions, and extremely disordered thinking and behaviour that impairs daily functioning and can be disabling.

Autism spectrum disorder (ASD) is a developmental disability caused by differences in the brain. People with ASD often have problems with social communication and interaction, and restricted or repetitive behaviours or interests. People with ASD may also have different ways of learning, moving, or paying attention.

### Diseases Characterised by Widespread Pathological Changes Throughout the CNS

### Neurogenerative Diseases not Elsewhere Listed

Lewy body disease, Multiple System Atrophy, Corticobasal degeneration, Hypertrophic Olivary Degeneration, and Leukodystrophies including Alexander disease, Autosomal dominant leukodystrophy with autonomic diseases, Canavan disease, Cerebrotendinous xanthomatosis, Childhood ataxia with central nervous system hypomyelination, Krabbe disease, Metachromatic leukodystrophy, Pelizaeus-Merzbacher disease, Refsum disease (adult or infantile), Rett syndrome and Progressive supranuclear palsy, hypomyelination with atrophy of the basal ganglia and cerebellum and essential tremor.

Multiple system atrophy is characterised by degeneration of the basal ganglia, inferior olivary nucleus and cerebellum resulting in autonomic dysfunction, tremor, bradykinesia, rigidity, postural instability and ataxia.

Pelizaeus-Merzbacher disease is an X-linked recessive leukodystrophy caused by a mutation in the proteolipid gene. This results in abnormalities throughout the white matter of brain and spinal cord.

Hypomyelination With Atrophy of the Basal Ganglia and Cerebellum is a rare genetic disorder which causes progressive degeneration of the basal ganglia and cerebellum. It is a type of leukodystrophy caused by a mutation in the TUBB4A gene.

### Enzyme Deficiency Disorders

Lysosomal Storage diseases, Tay Sachs Disease, Sandhoff Disease, Neuronal Ceroid Lipofuscinosis including Batten disease, Hunter Syndrome, Hurler disease, Niemann-Pick disease, Neuroaxonal Dystrophy, and Gaucher's Disease and Sanfilippo syndrome (mucopolysaccharidosis type III).

Neuroinflammatory diseases include Multiple Sclerosis, neurosarcoidosis, encephalitis, prion diseases and neuromyelitis optica (Devic disease).

Neuropsychiatric disorders in addition to those elsewhere listed including Attention deficit hyperactivity disorder (ADHD), affective disorders, Asperger's syndrome and addiction.

Acquired neurological disorders including stroke, traumatic brain injury and spinal cord injury.

### Potential Gene Therapies for Example Neurological Diseases Delivered to the Pons and DCN by CED Via a Trans-Middle Cerebellar Peduncle Trajectory

### Potential Gene Therapies for Amyotrophic Lateral Sclerosis (ALS):

Several approaches to gene therapy for ALS are being researched, and they target different aspects of the disease. Some potential gene therapy strategies for ALS include:
1. GDNF (Glial cell line-Derived Neurotrophic Factor): GDNF is a protein that supports the survival of nerve cells. Gene therapy involving the delivery of GDNF aims to protect motor neurons from degeneration in ALS.
2. IGF-1 (Insulin-like Growth Factor 1): IGF-1 has neuroprotective effects and may help promote motor neuron survival. Gene therapy delivering IGF-1 aims to support the survival of motor neurons in ALS.
3. SOD1 (Superoxide Dismutase 1): Mutations in the SOD1 gene are associated with some cases of familial ALS. Gene therapy targeting SOD1 aims to reduce the toxic effects of mutant SOD1 protein on motor neurons.
4. ALS2 (Alsin Rho Guanine Nucleotide Exchange Factor ALS2): Mutations in the ALS2 gene are linked to juvenile-onset forms of ALS. Gene therapy targeting ALS2 aims to address the underlying genetic cause of ALS.
5. C9orf72 (Chromosome 9 Open Reading Frame 72): Expansions of the C9orf72 gene are the most common genetic cause of familial ALS. Gene therapy approaches targeting C9orf72 aim to reduce the toxic effects of the expanded repeat sequences.

### Potential Gene Therapies for Friederich's Ataxia (FA):

Friedreich's Ataxia (FA) is a genetic disorder caused by mutations in the FXN (frataxin) gene. Frataxin is involved in mitochondrial function, and its deficiency leads to mitochondrial dysfunction and oxidative stress, particularly affecting the nervous system and heart.

Potential gene therapy approaches for Friedreich's Ataxia aim to address the underlying frataxin deficiency. Some strategies being explored include:
1. Frataxin Gene Replacement: Gene therapy techniques could involve delivering a functional copy of the FXN gene to cells affected by Friedreich's Ataxia. This could potentially restore frataxin expression and mitigate the symptoms of the disease.
2. Frataxin Gene Augmentation: Another approach is to enhance the expression of the remaining functional FXN gene in cells. This could involve techniques such as gene editing or using viral vectors to increase frataxin production.
3. Small Molecule Therapies: While not strictly gene therapy, small molecule compounds are being investigated to upregulate frataxin expression or enhance mitochondrial function in Friedreich's Ataxia.
4. Antioxidant Therapies: Given the role of oxidative stress in Friedreich's Ataxia pathology, antioxidant therapies are also being explored as potential treatments.

### Potential Gene Therapies for Spinocerebellar Ataxia Type 1 (SCA1)

Spinocerebellar Ataxia Type 1 (SCA1) is a genetic disorder caused by the expansion of a CAG repeat in the ATXN1 gene, leading to the production of a mutant ataxin-1 protein. This mutant protein disrupts normal cellular functions, particularly affecting the cerebellum and causing progressive degeneration of neurons.

While gene therapy for SCA1 is still in the preclinical and early clinical stages, there are several potential transgenes and approaches being explored:
1. Silencing of Mutant ATXN1: One approach is to use RNA interference (RNAi) or antisense oligonucleotides (ASOs) to selectively silence the expression of the mutant ATXN1 gene. This involves delivering small RNA molecules that target and degrade the mutant RNA, thereby reducing the production of the toxic mutant ataxin-1 protein.
2. Gene Editing: CRISPR-Cas9 and other gene editing technologies hold promise for correcting the genetic mutation underlying SCA1. Gene editing tools can be used to precisely edit the DNA sequence of the ATXN1 gene, either by removing the expanded repeat or by replacing it with the correct sequence.
3. Transgene Delivery: Another approach involves delivering a normal copy of the ATXN1 gene to affected cells. This could be achieved using viral vectors, such as adeno-associated viruses (AAVs), to deliver the transgene to the cerebellum and other affected brain regions. The normal ATXN1 gene would then produce functional ataxin-1 protein, potentially mitigating the symptoms of SCA1.
4. Neuroprotective Factors: In addition to targeting the mutant ATXN1 gene, gene therapy strategies for SCA1 may involve delivering neuroprotective factors or molecules that promote cell survival and function in the cerebellum and other affected regions of the brain.

### Potential Gene Therapies for Ceroid Lipofuscinosis Neuronal 5 (CLN-5):

CLN5, or Ceroid Lipofuscinosis Neuronal 5, is a form of neuronal ceroid lipofuscinosis, a group of rare inherited neurodegenerative disorders characterized by the accumulation of lipopigments in cells, particularly neurons. Currently, there are no specific gene therapy treatments approved for CLN5, but research is ongoing to develop potential therapies.

Since CLN5 is a genetic disorder caused by mutations in the CLN5 gene, potential gene therapy approaches would likely involve addressing the underlying genetic defect. Some strategies that could be explored include:
1. Gene Replacement Therapy: This involves delivering a functional copy of the CLN5 gene to affected cells. This could be achieved using viral vectors, such as adeno-associated viruses (AAVs), to deliver the normal CLN5 gene to neurons in the brain. Once delivered, the normal CLN5 gene would produce functional CLN5 protein, potentially correcting the underlying genetic defect and reducing the accumulation of lipopigments.
2. Gene Editing: CRISPR-Cas9 and other gene editing technologies could be used to correct the genetic mutation in the CLN5 gene. Gene editing tools can be used to precisely edit the DNA sequence of the CLN5 gene in affected cells, either by correcting the mutation or by replacing the defective gene with a normal copy.
3. Enhancing Autophagy and Lysosomal Function: Since CLN5 is involved in lysosomal function, another potential gene therapy approach is to enhance autophagy and lysosomal function in affected cells. This could involve delivering genes encoding lysosomal enzymes or proteins that promote autophagy to neurons in the brain, helping to clear accumulated lipopigments and alleviate symptoms.
4. Neuroprotective Factors: Gene therapy strategies for CLN5 could also involve delivering neuroprotective factors or molecules that promote cell survival and function in the brain. This could help to mitigate neuronal loss and slow the progression of the disease.
   Potential Gene Therapies for Niemann-Pick C1: Niemann-Pick disease type C1 (NPC1) is a rare lysosomal storage disorder caused by mutations in the NPC1 gene, which leads to impaired cholesterol trafficking within cells. There are currently no approved gene therapies for NPC1, but several approaches are being explored:
1. NPC1 Gene Replacement Therapy: Gene therapy can involve delivering a functional copy of the NPC1 gene to affected cells. This approach aims to restore normal NPC1 protein function, thereby correcting the underlying genetic defect. Viral vectors, such as adeno-associated viruses (AAVs) or lentiviruses, can be used to deliver the normal NPC1 gene to cells in the brain and other affected tissues.
2. Gene Editing: CRISPR-Cas9 and other gene editing technologies offer the potential to correct the genetic mutation in the NPC1 gene. Gene editing tools can be used to precisely edit the DNA sequence of the NPC1 gene in affected cells, either by correcting the mutation or by replacing the defective gene with a normal copy.
3. Cholesterol Metabolism Modulation: Since NPC1 affects cholesterol trafficking within cells, gene therapy approaches could aim to modulate cholesterol metabolism to reduce its accumulation in lysosomes. This could involve delivering genes encoding enzymes or proteins involved in cholesterol metabolism to affected cells, helping to restore normal cholesterol levels and alleviate symptoms.
4. Lysosomal Function Enhancement: NPC1 primarily affects lysosomal function, so gene therapy strategies could involve enhancing lysosomal function in affected cells. This could be achieved by delivering genes encoding lysosomal enzymes or proteins that promote lysosomal biogenesis and function, helping to clear accumulated lipids and alleviate symptoms.
5. Neuroprotective Factors: Gene therapy approaches for NPC1 could also involve delivering neuroprotective factors or molecules that promote cell survival and function in the brain. This could help to mitigate neuronal loss and slow the progression of neurological symptoms associated with the disease.

### Potential Gene Therapies for Multiple System Atrophy (MSA):

Multiple System Atrophy (MSA) is a rare neurodegenerative disorder characterized by the progressive degeneration of certain nerve cells in the brain, leading to symptoms such as autonomic dysfunction, movement disorders, and impaired coordination. Currently, there are no approved gene therapy treatments for MSA. However, research into potential gene therapy approaches is ongoing. Here are some potential strategies:
1. Neurotrophic Factors: Gene therapy could involve delivering genes encoding neurotrophic factors, such as glial cell line-derived neurotrophic factor (GDNF) or brain-derived neurotrophic factor (BDNF). These factors promote the survival and function of neurons, potentially slowing the progression of neurodegeneration in MSA.
2. Alpha-synuclein Regulation: MSA is characterized by the accumulation of alpha-synuclein aggregates in the brain. Gene therapy approaches could aim to regulate alpha-synuclein expression or clearance to reduce its accumulation and toxicity. This could involve delivering genes encoding proteins involved in alpha-synuclein degradation pathways or using RNA interference (RNAi) to silence alpha-synuclein expression.
3. Mitochondrial Function Enhancement: Dysfunction of mitochondria, the energy-producing organelles in cells, is implicated in MSA pathogenesis. Gene therapy strategies could involve enhancing mitochondrial function by delivering genes encoding mitochondrial proteins or factors that promote mitochondrial biogenesis and function.
4. Lysosomal Function Enhancement: MSA is also associated with impaired lysosomal function, leading to the accumulation of toxic substances within cells. Gene therapy approaches could aim to enhance lysosomal function by delivering genes encoding lysosomal enzymes or proteins that promote lysosomal biogenesis and function.
5. Neuroinflammation Modulation: Inflammation in the brain is thought to contribute to neurodegeneration in MSA. Gene therapy strategies could involve modulating neuroinflammatory pathways by delivering genes encoding anti-inflammatory factors or factors that promote the resolution of inflammation.

### Potential Gene Therapies for Progressive Supranuclear Palsy (PSP):

Progressive Supranuclear Palsy (PSP) is a rare neurodegenerative disorder characterized by the accumulation of abnormal tau protein in certain areas of the brain, leading to motor and cognitive impairments. Currently, there are no approved gene therapy treatments for PSP. However, research is ongoing, and several potential gene therapy approaches are being explored. Here are some possibilities:
1. Tau Protein Modulation: PSP is characterized by the accumulation of abnormal tau protein in neurons. Gene therapy approaches could aim to modulate tau protein expression or clearance to reduce its accumulation and toxicity. This could involve delivering genes encoding proteins involved in tau degradation pathways or using RNA interference (RNAi) to silence tau expression.
2. Neurotrophic Factors: Gene therapy could involve delivering genes encoding neurotrophic factors, such as glial cell line-derived neurotrophic factor (GDNF) or brain-derived neurotrophic factor (BDNF). These factors promote the survival and function of neurons, potentially slowing the progression of neurodegeneration in PSP.
3. Lysosomal Function Enhancement: Impaired lysosomal function is implicated in PSP pathogenesis, leading to the accumulation of toxic substances within cells. Gene therapy approaches could aim to enhance lysosomal function by delivering genes encoding lysosomal enzymes or proteins that promote lysosomal biogenesis and function.
4. Mitochondrial Function Enhancement: Dysfunction of mitochondria, the energy-producing organelles in cells, is implicated in PSP. Gene therapy strategies could involve enhancing mitochondrial function by delivering genes encoding mitochondrial proteins or factors that promote mitochondrial biogenesis and function.
5. Neuroinflammation Modulation: Inflammation in the brain is thought to contribute to neurodegeneration in PSP. Gene therapy strategies could involve modulating neuroinflammatory pathways by delivering genes encoding anti-inflammatory factors or factors that promote the resolution of inflammation.

### Potential Gene Therapies for Rett Syndrome:

Rett syndrome is a rare genetic neurological disorder caused by mutations in the MECP2 gene, which plays a crucial role in the development and function of the nervous system. Currently, there are no approved gene therapy treatments for Rett syndrome, but research is ongoing, and several potential gene therapy approaches are being explored. Here are some possibilities:
1. MECP2 Gene Replacement Therapy: Rett syndrome is primarily caused by mutations in the MECP2 gene, which leads to reduced levels or loss of functional MECP2 protein. Gene therapy approaches could involve delivering a functional copy of the MECP2 gene to affected cells, aiming to restore normal MECP2 protein levels and function. Viral vectors, such as adeno-associated viruses (AAVs), are commonly used to deliver genes to cells in the brain.
2. MECP2 Gene Editing: Gene editing technologies, such as CRISPR-Cas9, offer the potential to correct the genetic mutation in the MECP2 gene directly. Gene editing tools can be used to precisely edit the DNA sequence of the MECP2 gene in affected cells, either by correcting the mutation or by replacing the defective gene with a normal copy.
3. MECP2 Protein Modulation: Gene therapy approaches could also aim to modulate MECP2 protein expression or function. This could involve delivering genes encoding factors that regulate MECP2 expression levels or enhance the stability and activity of MECP2 protein.
4. Neurotrophic Factors: Rett syndrome is associated with abnormalities in brain development and function. Gene therapy could involve delivering genes encoding neurotrophic factors, such as brain-derived neurotrophic factor (BDNF) or insulin-like growth factor 1 (IGF-1), which promote the survival, growth, and function of neurons.
5. Synaptic Function Enhancement: Rett syndrome is characterized by abnormalities in synaptic function, the communication between neurons. Gene therapy strategies could aim to enhance synaptic function by delivering genes encoding proteins involved in synaptic transmission and plasticity.

### Potential Gene Therapies for Sanfillipo Syndrome:

Sanfilippo syndrome, also known as mucopolysaccharidosis type III (MPS III), is a group of rare genetic disorders caused by mutations in genes encoding enzymes involved in the breakdown of glycosaminoglycans (GAGs). There are four subtypes of Sanfilippo syndrome (A, B, C, and D), each resulting from mutations in different genes.

Currently, there are no approved gene therapy treatments for Sanfilippo syndrome. However, research is ongoing, and several potential gene therapy approaches are being explored. Here are some possibilities:
1. Enzyme Replacement Therapy (ERT): Enzyme replacement therapy involves delivering functional copies of the deficient enzyme to affected cells or tissues. In the case of Sanfilippo syndrome, this would involve delivering the enzyme deficient in the particular subtype of the disease (e.g., heparan N-sulfatase for MPS IIIA, alpha-N-acetylglucosaminidase for MPS IIIB). ERT is already used to treat some other types of mucopolysaccharidoses (MPS).
2. Gene Replacement Therapy: Gene therapy approaches could involve delivering functional copies of the mutated gene to affected cells. This could be achieved using viral vectors, such as adeno-associated viruses (AAVs), to deliver the normal gene to cells in the brain and other affected tissues. For example, in MPS IIIA, gene therapy would involve delivering the SGSH gene, and in MPS IIIB, it would involve delivering the NAGLU gene.
3. Gene Editing: CRISPR-Cas9 and other gene editing technologies offer the potential to correct the genetic mutation directly. Gene editing tools can be used to precisely edit the DNA sequence of the mutated gene in affected cells, either by correcting the mutation or by replacing the defective gene with a normal copy.
4. Enhancing Cellular Clearance Mechanisms: Sanfilippo syndrome is characterized by the accumulation of GAGs within lysosomes due to deficient enzyme activity. Gene therapy strategies could involve enhancing cellular clearance mechanisms, such as autophagy or lysosomal function, to help clear accumulated GAGs from cells.
5. Neuroprotective Factors: Sanfilippo syndrome affects the central nervous system, leading to progressive neurological deterioration. Gene therapy approaches could involve delivering genes encoding neuroprotective factors or molecules that promote cell survival and function in the brain.

### Potential Gene Therapies for Alzheimer's Disease:

Alzheimer's disease is the most common form of dementia and is characterised by amyloid plaques, cerebral amyloid angiopathy and neurofibrillary tangles affecting the cerebral cortex responsible for language, reason and social behaviour as well as areas of brain important for memory function including the hippocampus: Potential gene therapy strategies include:
1. Amyloid-degrading enzymes including protective protein/cathepsin A and neprilysin, ECE and cathepsin B.
2. Neurotrophic factors: gene therapy could involve delivering genes encoding neurotrophic factors, such as nerve growth factor (NGF), brain-derived neurotrophic factor (BDNF), neurotrophin-3 (NT-3), or glial cell line-derived neurotrophic factor (GDNF). These factors promote the survival, growth, and function of neurons.
3. Antisense oligonucleotides to “silence” the gene for tau protein.
4. SiRNA to downregulate Alzheimer's associated proteins such as BACE1 and APP.
5. Modification of APOE function.

### Potential Gene Therapies for Spinal Cord Injury:

Treating spinal cord injuries (SCIs) with gene therapy is a complex endeavour due to the multifaceted nature of these injuries. However, several potential gene therapy approaches are being explored to promote nerve regeneration, reduce inflammation, and enhance functional recovery. Here are some of the transgenes that could be used:
1. Neurotrophic Factors: Gene therapy could involve delivering genes encoding neurotrophic factors, such as nerve growth factor (NGF), brain-derived neurotrophic factor (BDNF), neurotrophin-3 (NT-3), or glial cell line-derived neurotrophic factor (GDNF). These factors promote the survival, growth, and function of neurons, potentially enhancing nerve regeneration and functional recovery after SCI.
2. Anti-inflammatory Factors: Inflammation plays a significant role in secondary injury mechanisms following SCI. Gene therapy approaches could aim to modulate the inflammatory response by delivering genes encoding anti-inflammatory factors, such as interleukin-10 (IL-10), interleukin-1 receptor antagonist (IL-1RA), or tumor necrosis factor-alpha (TNF-α) inhibitors.
3. Extracellular Matrix Modulation: The extracellular matrix (ECM) provides structural support and guidance for axonal growth. Gene therapy strategies could involve delivering genes encoding ECM-modifying enzymes or proteins that promote ECM remodeling to facilitate axonal regeneration and tissue repair.
4. Neuronal Plasticity Enhancers: Gene therapy approaches could aim to enhance neuronal plasticity and rewiring of neural circuits following SCI. This could involve delivering genes encoding proteins involved in synaptic plasticity, axon guidance, or dendritic spine formation.
5. Neuroprotective Factors: Gene therapy could involve delivering genes encoding neuroprotective factors or molecules that promote cell survival and function in the injured spinal cord. This could help to mitigate secondary injury mechanisms and preserve neuronal function following SCI.
6. Cell Replacement Therapy: Gene therapy approaches could involve delivering genes encoding factors that promote the differentiation and integration of transplanted stem cells or neural precursor cells into the injured spinal cord, potentially leading to functional recovery.
7. Modulation of Apoptosis: Gene therapy strategies could aim to modulate apoptotic pathways to reduce cell death and promote cell survival following SCI. This could involve delivering genes encoding anti-apoptotic factors or inhibitors of pro-apoptotic proteins.

### Potential Gene Therapies for Spinal Muscular Atrophy:

Spinal Muscular Atrophy (SMA) is a genetic disorder caused by mutations in the SMN1 gene, leading to a deficiency in the survival motor neuron (SMN) protein. Gene therapies have emerged as promising treatments for SMA, particularly targeting the underlying genetic cause of the disease. Here are the gene therapies used for treating different types of SMA:
1. SMA Type 1: SMA type 1 is the most severe form of the disease, typically appearing in infancy. The primary gene therapy used for treating SMA type 1 is Onasemnogene abeparvovec (brand name Zolgensma). It is a one-time infusion that delivers a functional copy of the SMN1 gene to replace the defective one. Zolgensma utilizes an adeno-associated virus (AAV) vector to deliver the corrective gene to motor neurons.
2. SMA Type 2: SMA type 2 usually manifests in infancy or early childhood and presents with less severe symptoms than type 1. While Zolgensma is primarily indicated for SMA type 1, its use has also been explored in SMA type 2 patients. However, other therapies like nusinersen (Spinraza) are more commonly used for SMA type 2. Nusinersen is an antisense oligonucleotide that modifies the splicing of SMN2 mRNA, increasing the production of functional SMN protein.
3. SMA Type 3: SMA type 3, also known as Kugelberg-Welander disease or juvenile SMA, typically appears later in childhood or adolescence and is milder compared to types 1 and 2. As of my last update, no specific gene therapy has been approved specifically for SMA type 3. Treatment for SMA type 3 often involves multidisciplinary care, physical therapy, and in some cases, medications to manage symptoms. However, ongoing research may lead to the development of gene therapies targeting this type of SMA in the future.

The non-malignant CNS disorder may be selected from neurodegenerative diseases, enzyme deficient conditions, neuroinflammatory diseases, acquired neurological injuries, or neurological disorders including neuropsychiatric conditions. For the avoidance of doubt, the non-malignant CNS disorder is not cancer.

The neurodegenerative disease may be selected from dementia, Lewy body disease, Alzheimer's disease, Huntington's disease, Amyotrophic Lateral Sclerosis (ALS), Multiple System Atrophy, Spinal muscular atrophy, Spino-cerebellar ataxia, Friedreich's Ataxia, Huntington's disease, Parkinson's disease, Parkinson's plus syndromes, Corticobasal degeneration, Hypertrophic Olivary Degeneration, and Leukodystrophies including Alexander disease, Autosomal dominant leukodystrophy with autonomic diseases, Canavan disease, Cerebrotendinous xanthomatosis, Childhood ataxia with central nervous system hypomyelination, Krabbe disease, Metachromatic leukodystrophy, Pelizaeus-Merzbacher disease, Refsum disease (adult or infantile), Rett syndrome and Progressive supranuclear palsy.

The enzyme deficient condition may be selected from Lysosomal Storage diseases, Tay Sachs Disease, Sandhoff Disease, Neuronal Ceroid Lipofuscinosis, Hunter Syndrome, Hurler disease, Niemann-Pick disease, Neuroaxonal Dystrophy, Gaucher's Disease and Sanfilippo syndrome (mucopolysaccharidosis type III).

The neuroinflammatory disease may be selected from Multiple Sclerosis, neurosarcoidosis, encephalitis, prion diseases and neuromyelitis optica (Devic disease).

The acquired neurological injury may be stroke, traumatic brain injury or spinal cord injury.

The neurological disorder may be epilepsy, essential tremor or limbic encephalitis.

The gene or RNA therapy vector may be administered in the form of a pharmaceutical composition, which is preferably sterile and may comprise one or more pharmaceutically acceptable carriers or excipients. Suitable carriers and excipients will be familiar to the skilled person and may be optimised in line with the intended route of delivery. For example, suitable pharmaceutical compositions may include buffers, binders, preservatives, thickeners or antioxidants.

The gene or RNA therapy vector is preferably for administration by infusion for between 1 and 24 hours, especially for at least 1, 2, 3, 4, 5, 6, 7, 8, 9 or 10 hours and/or for less than 10, 9, 8, 7, 6, 5, 4, or 3 hours. It is preferably for infusion for around 5 hours or around 3 hours. In embodiments of the invention the gene or RNA therapy vector may be for repeated daily infusions over a period of up to 6 days.

Whether or not the gene or RNA therapy vector is for administration for a number of consecutive days or for regular administration over a number of days, it may independently or additionally be for administration weekly, fortnightly, monthly, every six, eight, twelve or fifteen or more weeks. For example, a cycle of two or three days of infusions may be repeated every three months. Alternatively, it may be for administration in a series of cycles of infusions, with 6, 7, 8, 9, 10, 11 or 12 months between the end of a first cycle of infusions and the next cycle of infusions.

The non-malignant CNS disorder is preferably treated in a subject in need thereof. The subject is preferably a mammal, more preferably a primate, especially a human and may be a paediatric or geriatric patient.

The present invention additionally provides methods for treating for treating non-malignant CNS disorders comprising infusing a gene or RNA therapy vector into the pons via CED in accordance with the embodiments of the invention described above.

### BRIEF DESCRIPTION OF THE DRAWINGS

The invention will now be described in detail, by way of example only, with reference to the figures.

FIG. 1 shows an axial diagram (A) and histological specimen (B) through the pons, which demonstrate the structures within the pons at the axial level of the middle cerebellar peduncle. Anteriorly at this level are descending motor corticospinal and corticobulbar tracts. Posterior to these are the transverse pontine (ponto-cerebellar) fibres which connect the cerebral cortex the contralateral cerebellar cortex via the middle cerebellar peduncle. More posterior to these lies the medial lemniscus, which carries the second-order neurons of the sensory dorsal columns which has important roles in proprioception, vibration sense, fine touch and 2-point discrimination. Throughout the pons are important nuclei from which cranial nerves originate.

FIG. 2 shows an axial view of pons, middle cerebellar peduncles and deep cerebellar nuclei (A). The pons is connected to the cerebellum via the middle cerebellar peduncles. At this level are the deep cerebellar nuclei (DCN) which have important roles in transmitting movement fibres from the cerebellum to the thalamus and motor cortex in order to optimise the accuracy and co-ordination of movements. Note: 1. Pons, 2. Middle cerebellar peduncle, 3. Nodulus, 4. Fourth ventricle, 5. Deep cerebellar nuclei (DCN). FIG. 2 B shows an axial diagram though the pons and the middle cerebellar peduncle at its more caudal level. This illustrates the relationship of the inferior cerebellar peduncle to the middle cerebellar peduncle in this region. The infusion of a gene therapy covering the via the middle cerebellar peduncle will also be distributed to the inferior cerebellar peduncle which carries spinocerebellar, inferior olivocerebellar, and vestibulocerebellar fibres that carry information essential for coordinated motor functioning and learning that would not be covered with an infusion confined to the pons.

FIG. 3 shows an exemplar illustration of bilateral transcerebellar cannula placement via the middle cerebellar peduncles, placing cannula steps at the interface of the DCN (5) with posterior white matter. The cannula trajectory traverses lateral to the DCN rather directly injecting into the DCN in order to prevent injury to these important nuclei (A). FIG. 3B shows an equivalent axial MRI image with a unilateral cannula infusion of AAV9-mCherry, co-infused with an MRI contrast agent, along the left middle cerebellar peduncle and pons. (B) shows a sagittal view of the trajectory and the infusion.

FIG. 4 shows a diagrammatic representation of the relationship of the DCN, Purkinje cells and inferior olive. The dentate nucleus is the largest of the DCN. Purkinje cells within the cerebellar cortex project to and inhibit the dentate nucleus which projects axons via the motor thalamus to the motor cortex. The inferior olivary nucleus is a source of climbing fibres to the Purkinje cells of the cerebellar cortex. These structures have important roles in motor coordination.

FIG. 5. Pre-infusion (left) and post-infusion (middle) axial MR images and sagittal image (right) confirms gadolinium distribution in the pons bilaterally.

FIG. 6. GFP expression measured by ELISA confirms expression in the ipsilateral motor cortex, contralateral motor cortex and spinal cord at 1 month post unilateral pontine infusion of AAV5-GFP.

FIG. 7. mCherry expression measured by ELISA confirms expression in the ipsilateral motor cortex, contralateral motor cortex and spinal cord at 1 month post unilateral infusion of AAV9-mCherry.

FIG. 8. Fluorescence microscopy confirms mCherry expression in the hypoglossal nuclei and inferior olivary nuclei of the medulla.

FIG. 9. Fluorescence microscopy confirms mCherry expression in the ventral horn cells of the spinal cord.

FIG. 10. mCherry expression measured in pg/ml by ELISA following unilateral (left pons) infusion confirms expression in contralateral and ipsilateral motor subsystems—orbitofrontal cortex, motor cortex, premotor cortex, caudate, putamen, motor thalamus, pons, origin of the trigeminal nerve, vestibular nucelus, hypoglossal nucleus, cerebellar cortex, dentate nucleus and anterior horn cells of spinal cord. Arbitrary values of 400 000 are ascribed to samples which exceeded the upper threshold of detection (log scale).

FIG. 11. mCherry expression measured in pg/ml by ELISA following unilateral (left pons) infusion confirms expression in contralateral and ipsilateral sensory subsystems—sensory cortex, auditory cortex, sensory thalamus, Clarkes column, dorsal column of spinal cord. Arbitrary values of 400 000 are ascribed to samples which exceeded the upper threshold of detection (log scale).

FIG. 12. mCherry expression measured in pg/ml by ELISA following unilateral (left pons) infusion confirms expression in contralateral and ipsilateral visual subsystems—optic tract, visual cortex and visual association cortex. Arbitrary values of 400 000 are ascribed to samples which exceeded the upper threshold of detection (log scale).

FIG. 13. mCherry expression measured in pg/ml by ELISA following unilateral (left pons) infusion confirms expression in contralateral and ipsilateral peripheral nerves and muscle—sciatic nerve, tibial nerve, dorsal root ganglia, cheek muscle and tongue.

FIG. 14. mCherry expression measured in pg/ml by ELISA following unilateral (left pons) infusion confirms expression in contralateral and ipsilateral limbic subsystems—amygdala, hippocampus and temporal lobe cortex.

FIG. 15. mCherry detection by DAB immunohistochemistry confirms expression in left and right orbitofrontal and prefrontal cortex. Histology (right sided images are shown with corresponding sections from the Wisconsin sheep brain atlas (left image).

FIG. 16. mCherry detection by DAB immunohistochemistry confirms expression in left and right primary motor and somatosensory cortex. Histology (right sided images are shown with corresponding sections from the Wisconsin sheep brain atlas (left image).

FIG. 17. mCherry detection by DAB immunohistochemistry confirms expression in left and right caudate and putamen. Histology (right sided images are shown with corresponding sections from the Wisconsin sheep brain atlas (left image).

FIG. 18. mCherry detection by DAB immunohistochemistry confirms expression in left and right temporal cortex, parietal cortex, hypothalamus and amygdala. Histology (right sided images are shown with corresponding sections from the Wisconsin sheep brain atlas (left image).

FIG. 19. mCherry detection by DAB immunohistochemistry confirms expression in lateral geniculate nucleus, optic tract, auditory cortex, parietal cortex, hippocampus, medial lemniscus and thalamus. Histology (right sided images are shown with corresponding sections from the Wisconsin sheep brain atlas (left image).

FIG. 20. mCherry detection by DAB immunohistochemistry confirms expression in left and right pons, superior colliculus, raphe nucleus, visual and visual association cortex. Histology (right sided images are shown with corresponding sections from the Wisconsin sheep brain atlas (left image).

FIG. 21. mCherry detection by DAB immunohistochemistry confirms expression in left and right cerebellar cortex, deep cerebellar nuclei, vestibular nuclei, inferior cerebellar peduncle and pyramid. Histology (right sided images are shown with corresponding sections from the Wisconsin sheep brain atlas (left image).

FIG. 22. mCherry detection by DAB immunohistochemistry confirms expression in medulla, cuneate and gracile nuclei, vagus nucleus, inferior olive, pyramid, medial longitudinal fasciculus and spinal trigeminal nucleus. Histology (right image) is shown with corresponding sections from the Wisconsin sheep brain atlas (left image).

FIG. 23. mCherry detection by DAB immunohistochemistry confirms expression in Clarke's column within the cervical spinal cord (left images), anterior horn cells of the thoracic spinal cord (middle images) and anterior horn cells of lumbar spinal cord (right images).

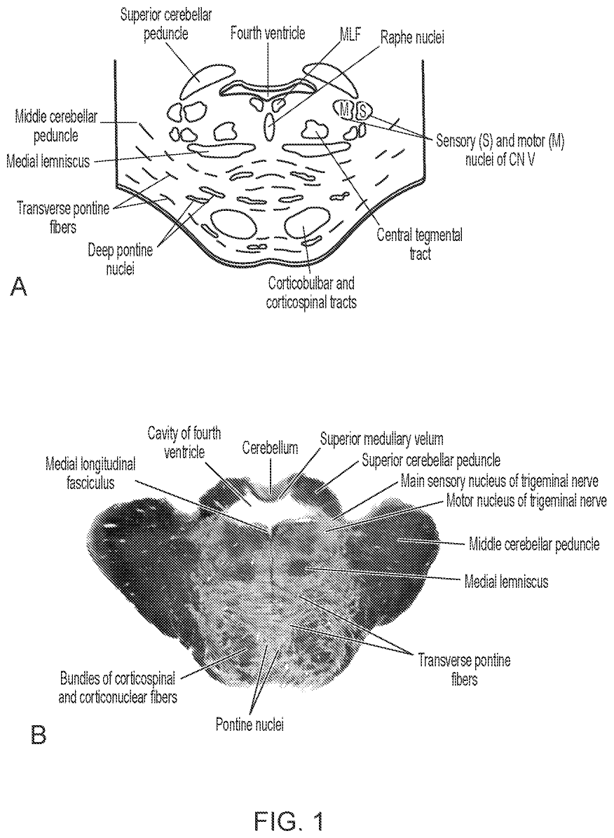

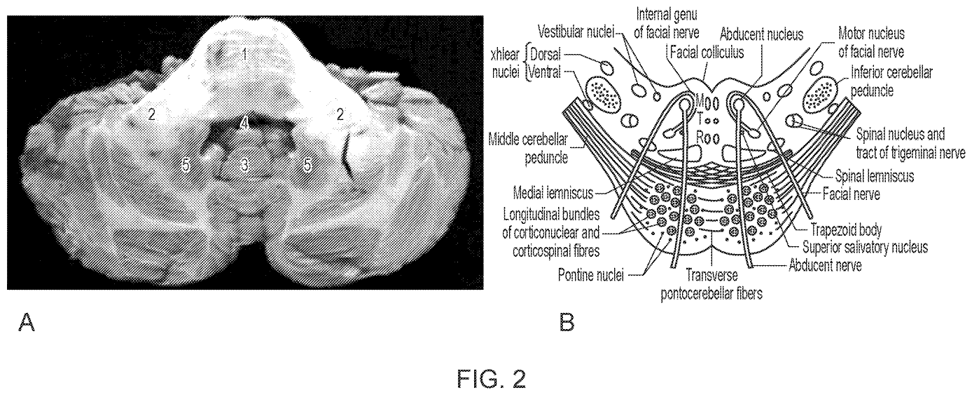

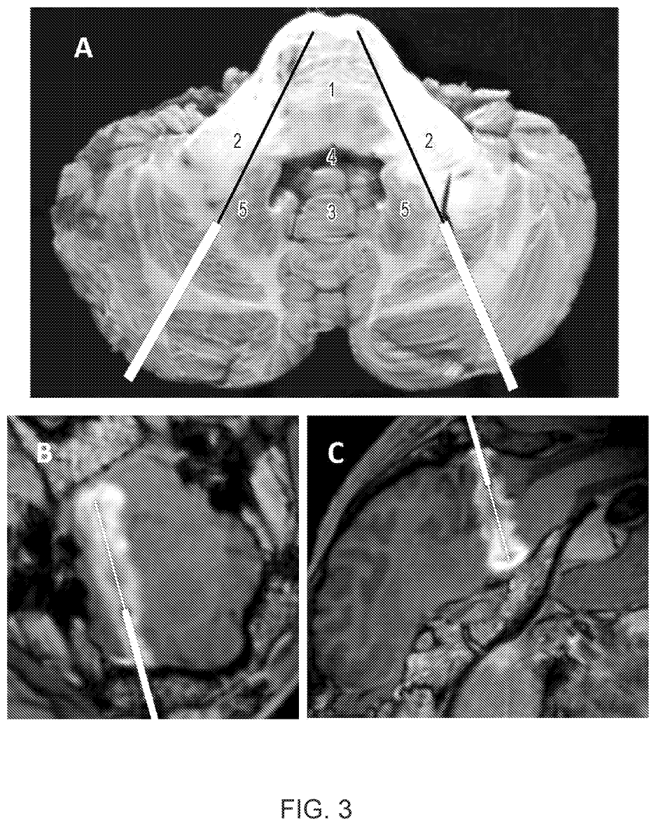

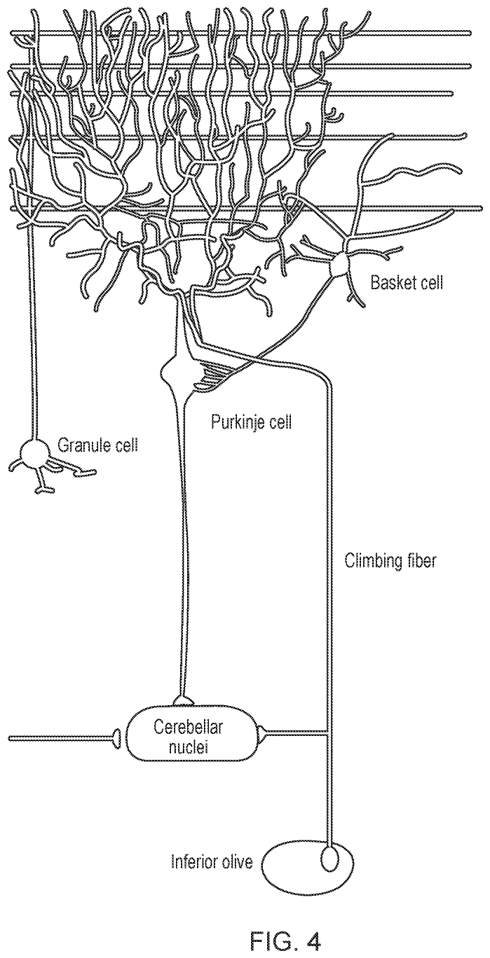

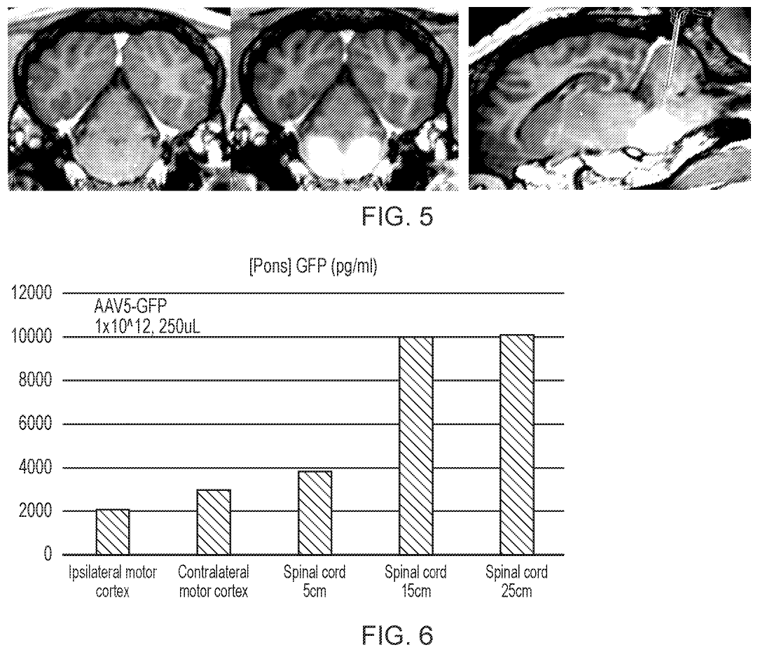

### EXAMPLES

### 1. Infusion of AAV9-mCherry and AAV5-GFP

All work was conducted in accordance with the Animals (Scientific Procedures) Act (1986) and with the authority of appropriate UK Home Office project and personal licences. The sheep model is particularly relevant as development of projections from the brainstem nuclei are the same as in other mammalian species (Stockx et al., 2007).

### Sheep Anaesthesia

The test subject was transferred into the induction room and administered with an intramuscular injection of Ketamine (10 mg/Kg) and Dexmedetomidine (15 mcg/kg) in the superior aspect of the neck. An intra-venous (IV) catheter was positioned in an ear vein. The induction of general anaesthesia was achieved via IV infusion of Propofol at 1.0 mg/Kg boluses until loss of palpebral reflex. After induction, the airway was secured with appropriate size endotracheal tube. Anaesthesia was maintained by Isoflurane in oxygen 1-2.5%. The animal was mechanically ventilated using a target of Vt of 10-20 ml/Kg and a RR of 10-20 breaths per minute. Settings were adjusted to maintain ET-CO₂ between 35 and 45 mmHg. Before proceeding with the experiment, the animal was secured to the surgical table and positioned in sternal position.

An accurate shaving and cleaning of the skin in the neck region (or groin region) was achieved in preparation for the surgical field. The monitoring devices included:
an invasive arterial blood pressure catheter
a peripheral oxygen saturation probe
a core temperature probe
a central venous line inserted in the jugular vain

### Surgical Preparation and Pre-Surgery:

A custom-made Ovine model fixation frame was used to immobilise the subject's skull within the preparation room and throughout the image acquisition, surgical and infusion procedures. Localisation of the anatomy was performed using a dedicated fiducial frame and built in co-registration module fitted to the fixation frame prior to transfer to the MRI scanner.

### MRI Scanning

Surgical planning and real-time sequential scans of intracranial infusions were acquired on a 3T MRI unit (Prisma, Siemens). Body surface receiver coils were used in combination with the Ovine fixation and fiducial platform.

### Detailed Trajectory Planning and Surgery

On completion of MRI imaging, the subject was transferred to the surgical suite for bilateral refluxing cannula implantation into the pons.

Following the MRI acquisition, the anaesthetized sheep was moved to the operating theatre and positioned on the operating table for insertion of cannulae into the pons bilaterally. The head was prepared and draped and a CRW stereoguide was set to the coordinates of the first target. Through a 10-20 mm linear scalp incision a profiled hole is made through the skull that is co-axial with the trajectory using custom made tooling and a setting jig. A guide hub was delivered into the profiled hole and secured with its self-tapping thread. The guide tube, cut to length was inserted through the guide hub followed by the primed cannula, cut to length, which is locked into the hub with its threaded stop. The cannula comprised a continuous 2-meter length of PEEK tube (0.5 mm OD, 0.127 mm ID) with a threaded stop 5 cm from its distal end and with a proximal connection to a 500 μl Hamilton® syringe. The dead-volume of the catheter and infusion line was 25 μl.

The CRW coordinates were then set for the second target and the second cannula is now inserted and secured as previously described.

### Infusions

Infusions of AAV vector in aCSF with 0.2% Gadolinium were performed as described in Table 1. The reporter genes mCherry and GFP were chosen to allow analysis with ELISA, fluorescent microscopy and DAB immunohistochemistry.

TABLE 1
Infusions

| | | | Infused | | Flow rate |
|---|---|---|---|---|---|
| Test | | | titre | Volume | (microlitres/ |
| subject | Target | Infusate | (Vg/ml) | (microlitres) | min) |
| 1 | Right | AAV9- | 1 × 10¹² | 250 | 3 |
| | pons | mCherry | | | |
| | Left | AAV5-GFP | 1 × 10¹² | 250 | 3 |
| | pons | | | | |
| 2 | Right | AAV9- | 1 × 10¹² | 250 | 3 |
| | pons | mCherry | | | |
| | Left | AAV9- | 1 × 10¹² | 250 | 3 |
| | pons | mCherry | | | |
| 3 | Left | AAV9- | 5 × 10¹² | 300 | 3 |
| | pons | mCherry | | | |

### Analyses

Immediate post-infusion MRI scans were performed in order to determine gadolinium distribution. Test subjects showed no adverse neurological, respiratory or cardiac signs throughout the surgery, infusion or recovery period. The subjects were terminated at one month post infusion and brain and spinal cord explanted. Explanted brain and spinal cord from subject 1 was immediately frozen for analysis of mCherry and GFP expression by ELISA. Explanted brain from subject 2 was formalin fixed for fluorescence microscopy.

The explanted brain from subject 3 was cut into 0.5 cm coronal sections. Alternate sections were immediately frozen of mCherry expression by ELISA. Remaining sections were formalin fixed for DAB immunohiostochemistry. The spinal cord was cut into 5 cm sections. The proximal 1 cm of each section was frozen for ELISA, whilst the remaining 4 cm was formalin fixed for DAB immunohistochemistry.

### Results—Post-Infusion MRI

Immediate post-infusion MRI confirmed gadolinium distribution throughout the pons (FIG. 6).

### Results—GFP ELISA: Test Subjects 1 & 2

At one-month post-infusion of AAV5-GFP into the left pons, GFP expression was identified in ipsilateral motor cortex, contralateral motor cortex, and spinal cord (FIG. 6).

Results—mCherry ELISA: Test Subjects 1 & 2

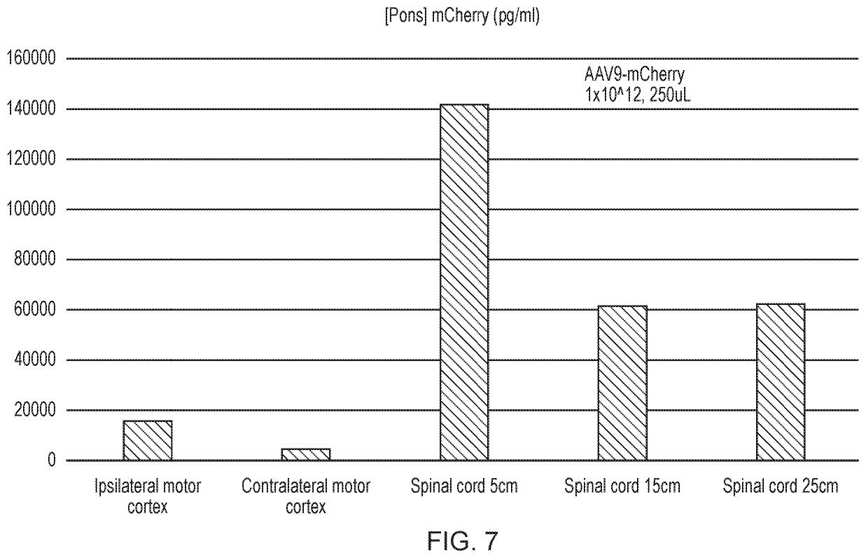

At one-month post-infusion of AAV9-mCherry into the right pons, mCherry expression was identified in ipsilateral motor cortex, contralateral motor cortex, and spinal cord (FIG. 7).

Results—mCherry Fluorescence Microscopy

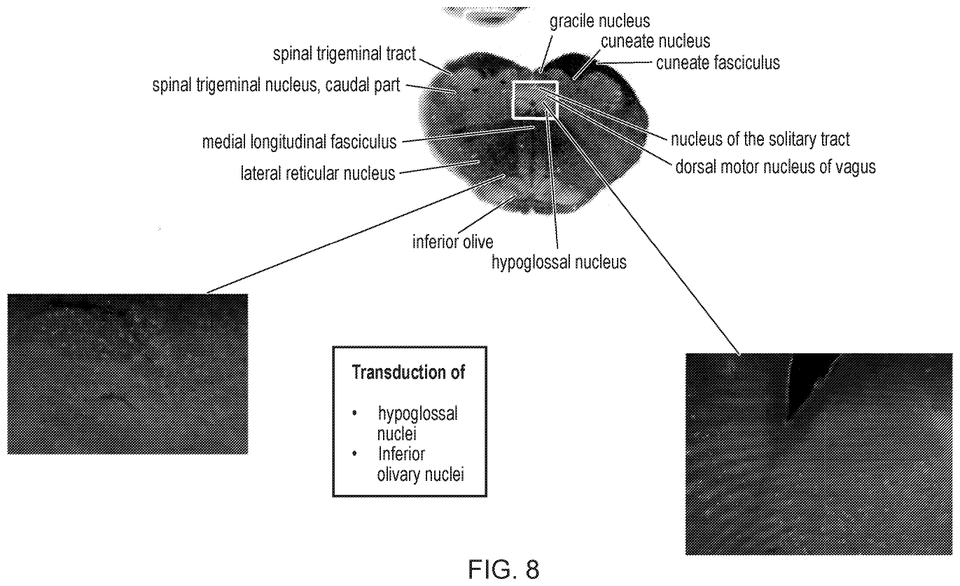

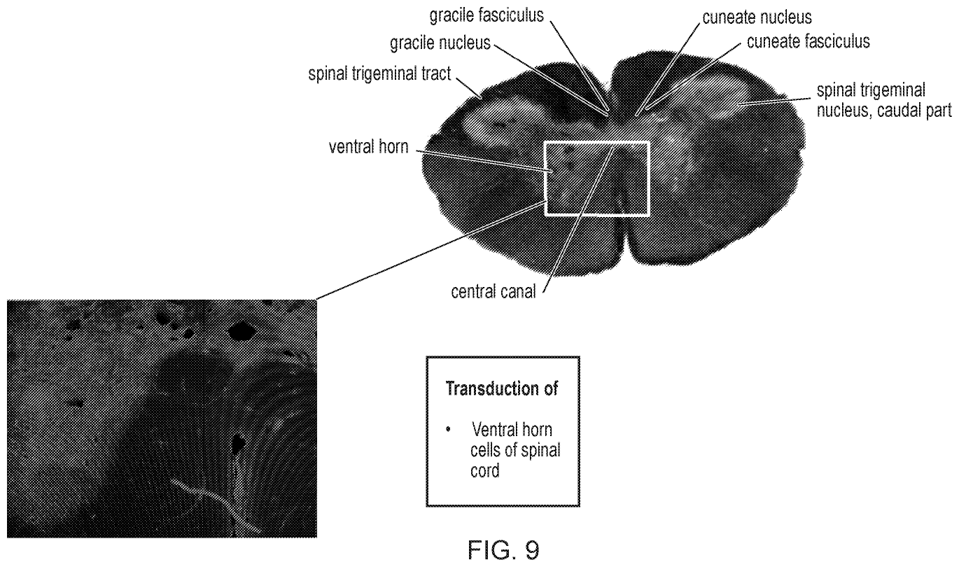

mCherry was visualised in the medulla within the inferior olivary nuclei and hypoglossal nuclei (FIG. 8), as well as the spinal cord (FIG. 9).

Results—mCherry ELISA: Test Subject 3 (Unilateral Pons Infusion)

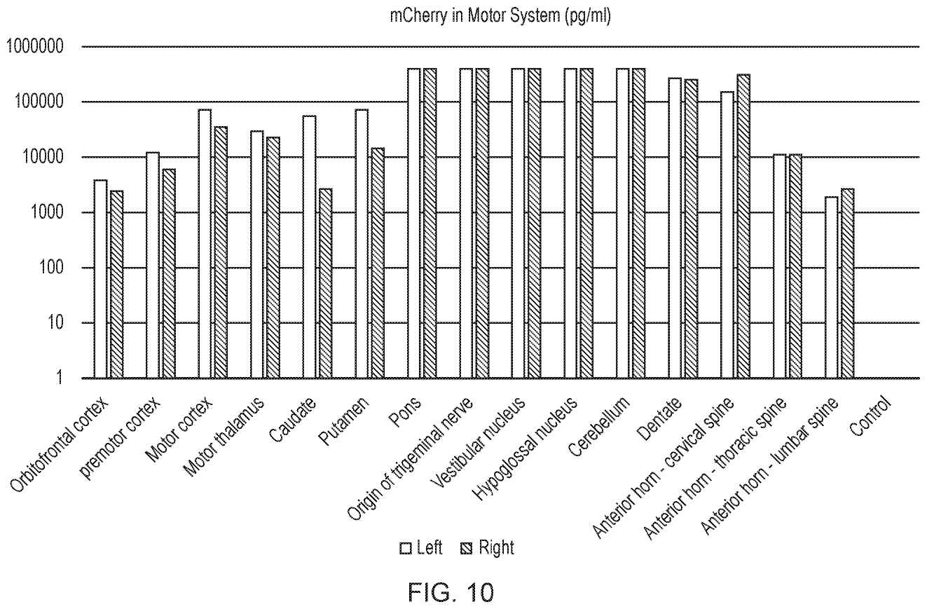

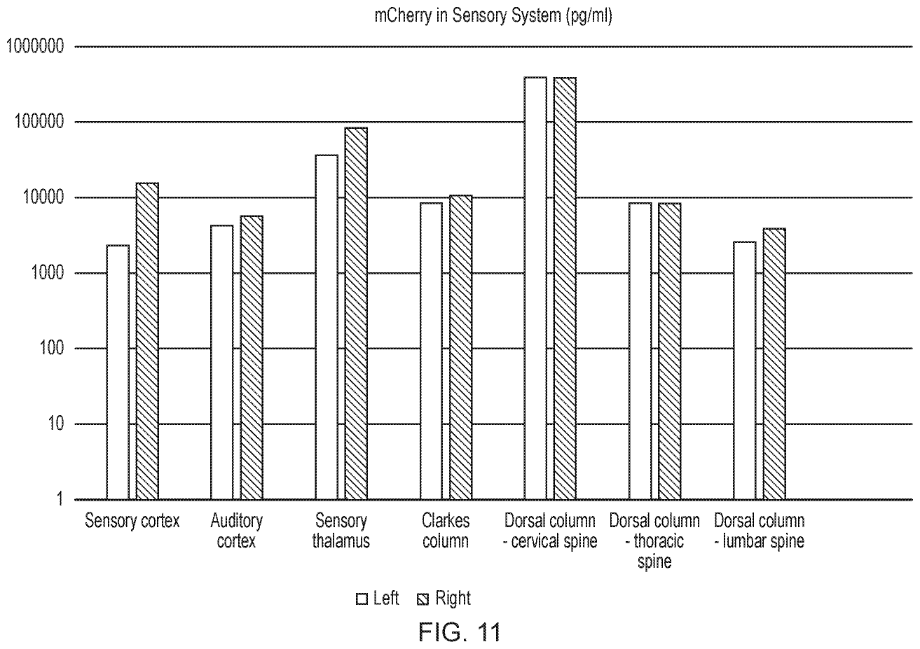

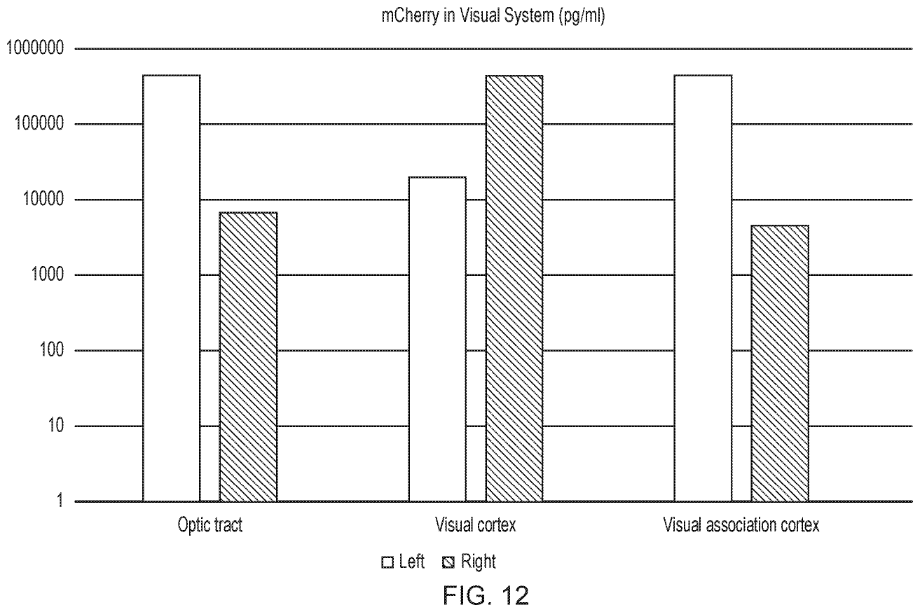

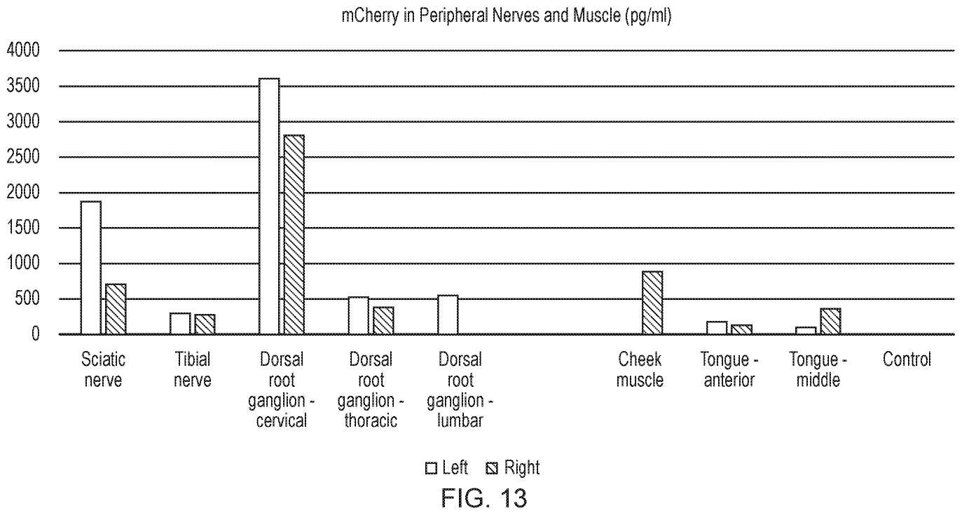

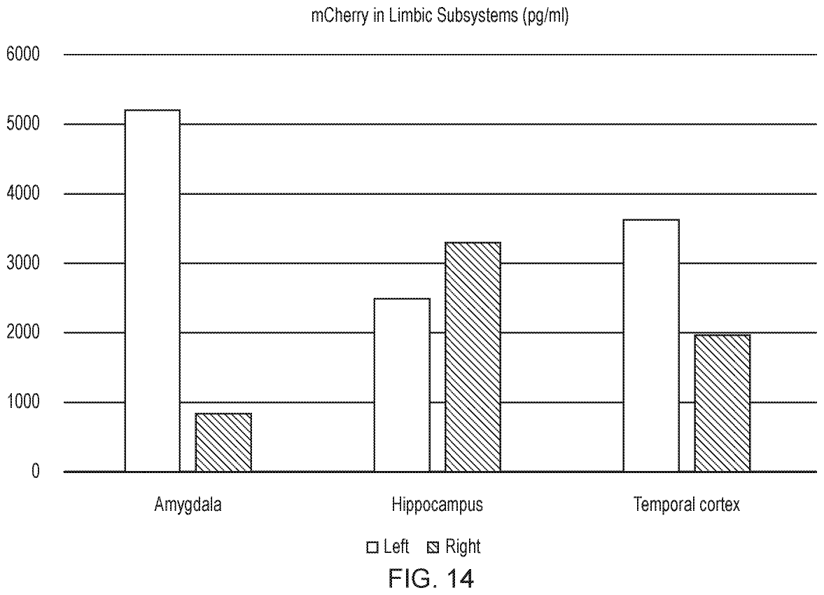

At one-month post-infusion of AAV9-mCherry into the left pons, mCherry expression was identified bilaterally in:
orbitofrontal, premotor and motor cortex, putamen, caudate, motor thalamus, vermis, origin of the trigeminal nerve, cerebellar cortex, dentate nucleus and anterior horn cells of cervical, thoracic and lumbar, spinal cord (FIG. 10).
sensory cortex, auditory cortex, Clarke's column and dorsal columns (FIG. 11).
Optic tract, visual cortex and visual association cortex (FIG. 12).
Sciatic nerve, tibial nerve, dorsal root ganglia, cheek muscle (right only) and tongue muscle (FIG. 13).
Hippocampus, amygdala and temporal cortex (FIG. 14).
   Results—mCherry DAB Immunohistochemistry: Test Subject 3 (Unilateral Pons Infusion)

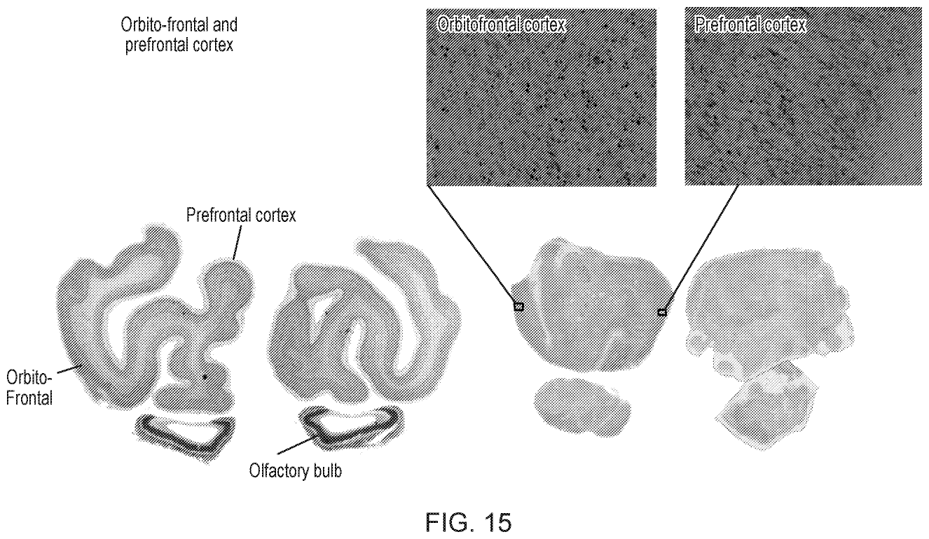

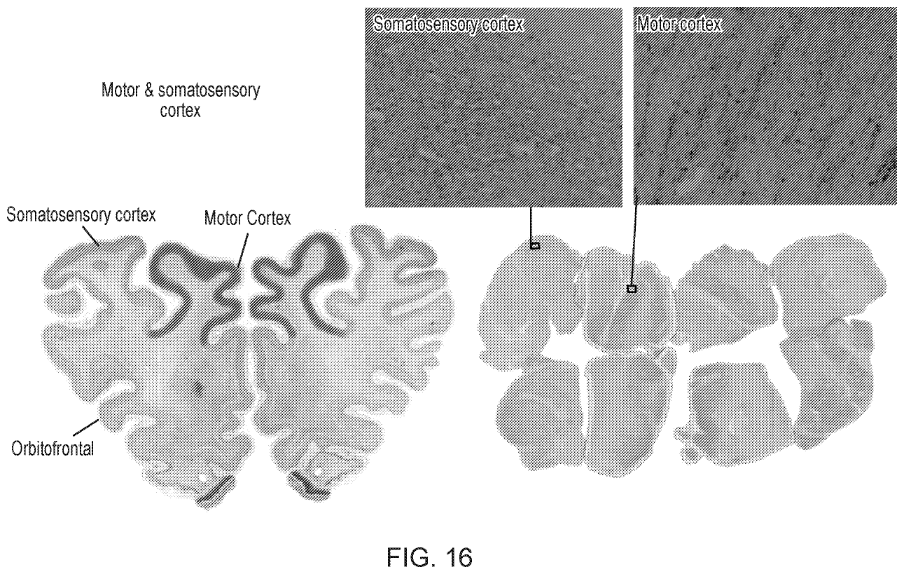

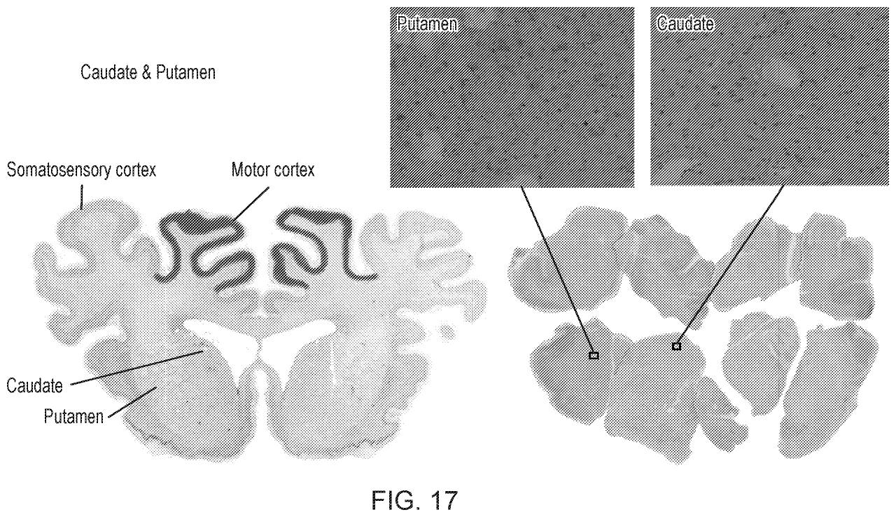

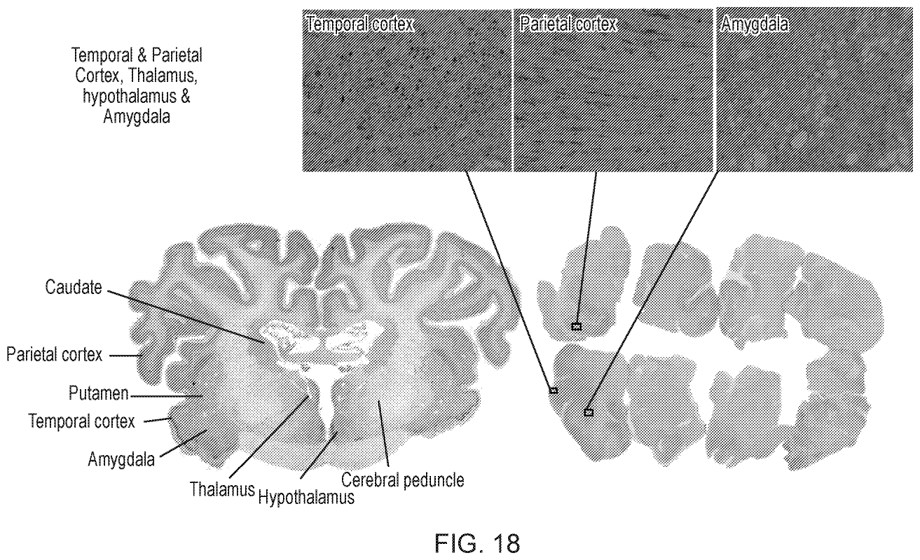

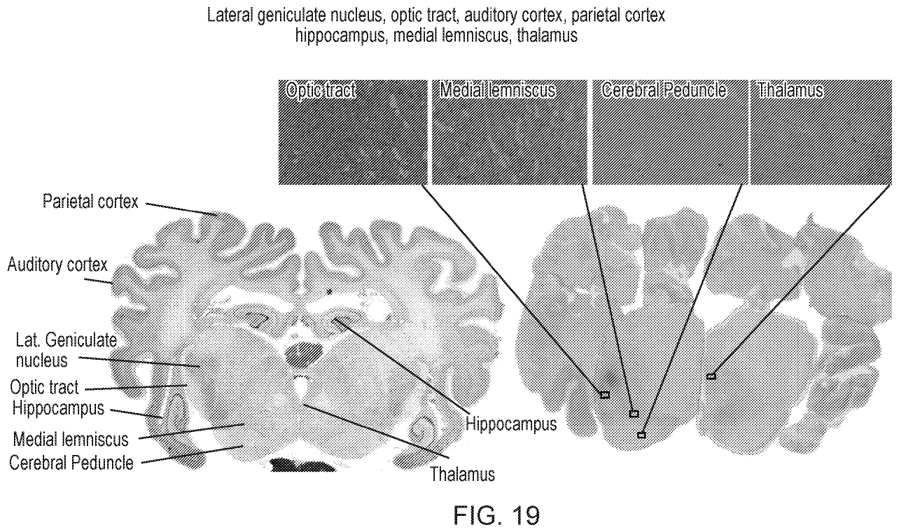

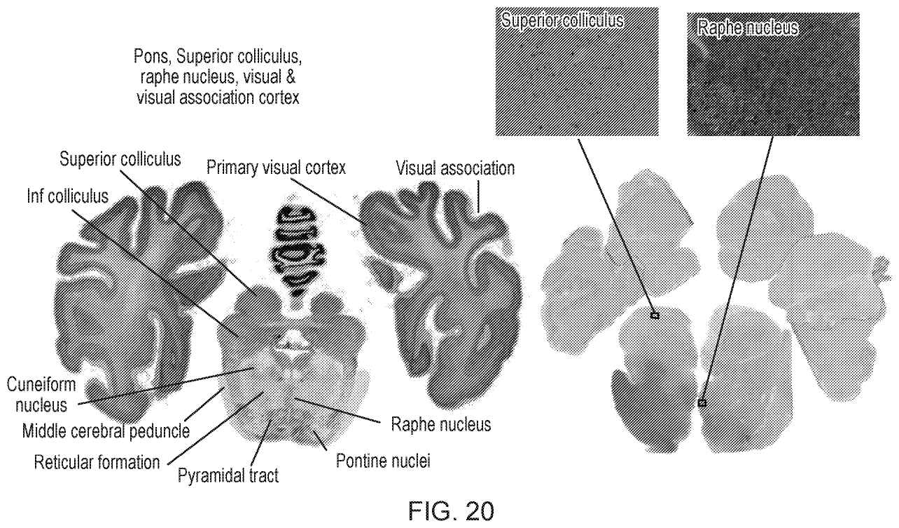

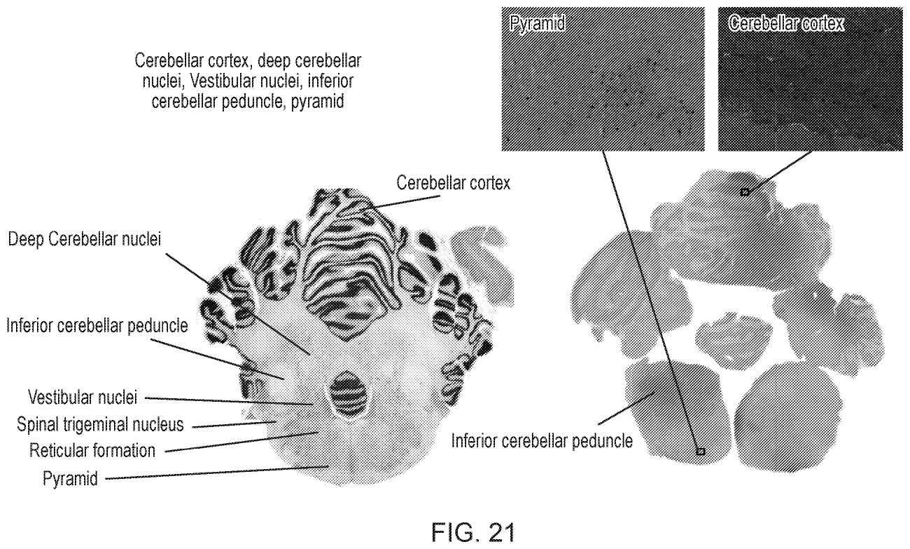

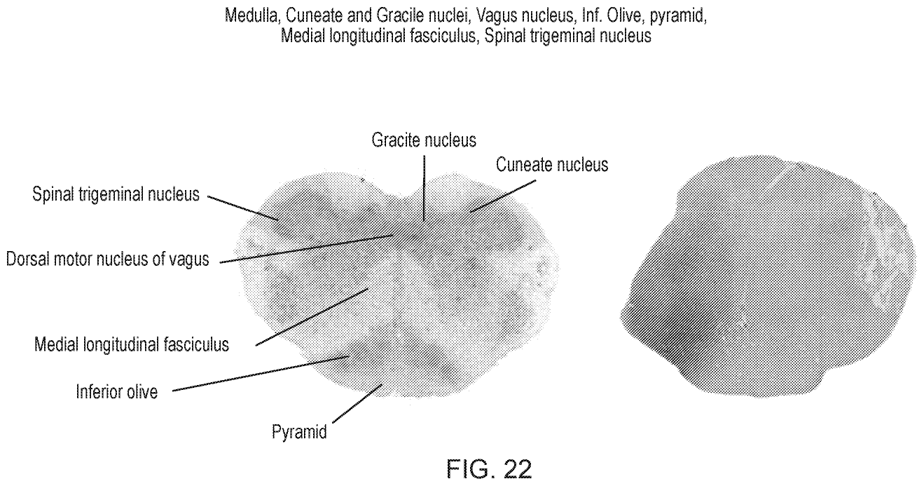

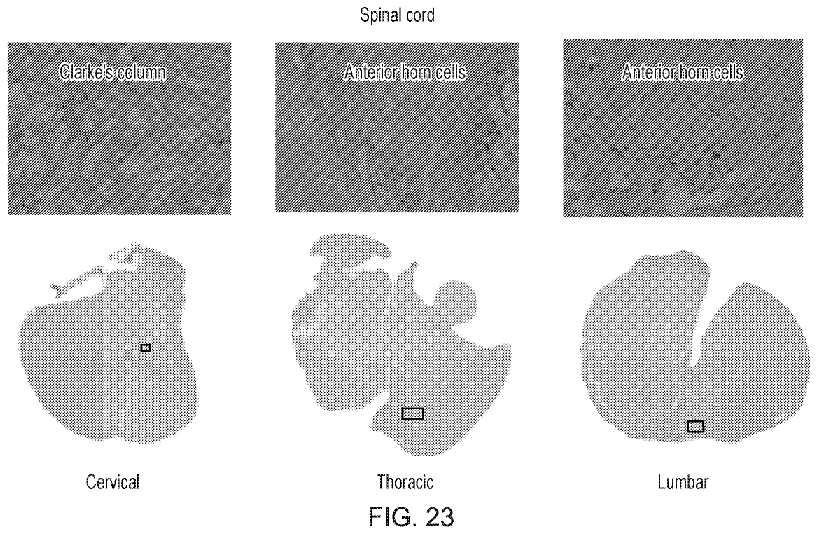

At one-month post-infusion of AAV9-mCherry into the left pons, mCherry expression was detected bilaterally in:
Orbitofrontal and pre frontal cortex (FIG. 15).
Motor and somatosensory cortex (FIG. 16).
Caudate and putamen (FIG. 17).
Temporal and parietal cortex, thalamus, hypothalamus and amygdala (FIG. 18).
Lateral geniculate nucleus, optic tract, auditoty cortex, parietal cortex, hippocampus, medial lemniscus and thalamus (FIG. 19).
Pons, superior colliculus, raphe nucleus, visual and visual association cortex (FIG. 20).
Cerebellar cortex, deep cerebellar nuclei, vestibular nuclei, inferior cerebellar peduncle and pyramid (FIG. 21).
Medulla, cuneate and gracile nuclei, vagus nuclei, inferior olive, pyramid, medial longitudinal fasciculus and spinal trigeminal nucleus (FIG. 22).
Clarke's column and anterior horn cells of the cervical, thoracic and lumbar spinal cord (FIG. 23).

### CONCLUSION

These data demonstrate that a single CED infusion of an adeno-associated viral vector to the pons through a transcerebellar trajectory results in gene expression in multiple components of the cerebral cortex, motor system and sensory and limbic systems required for modulation of motor activity including—the motor cortex, basal ganglia, sensory, auditory, visual and visual association cortex, hippocampus and amygdala, spinal cord, cranial nerve nuclei, inferior olivary nucleus, thalamus, cerebellum including dentate nucleus, as well as the anterior horn cells of the spinal cord, and also in the peripheral nervous system and musculature.

### REFERENCES

Kimura S & Harashima H., Pharmaceutics. 2020; 15; 12(12):1216
Bailey R M et al., Mol Ther Methods Clin Dev. 2018; 15; 9:160-171
Kells A P et al., Proc Natl Acad Sci USA. 2009; 106(7):2407-2411
Khateeb K et al., J Vis Exp. 2019; 23; (147)
Nieuwenhuis B et al., Gene Therapy 2021; 28:56-74
Barua N U et al., J Neurosci Methods. 2013; 220(1):1-8
Salegio E A et al., Hum Gene Ther. 2022; 33(1-2): 86-93
Bravo-Hernandez et al., Nat Med. 2020; 26(1):118-130
Salegio et al., Mol Ther. 2010; 18(8):1490-1495
Li et al., Neurosci. Bull. 2019; 35(5):909-920
Gill T, et al. J Neurosci Methods. 2013; 219(1):1-9
Panse S J, et al. J Med Eng Technol. 2010; 35(7-8):408-414.
Arrigo et al. Neuroscience. 2017; 343, P346-354.
McLachlan & Wilson. Fron Psychol. 2017; 8:265.
Stockx et al. BMC Neurosci. 2007; 8:40.

1-23. (canceled)

24. A method for treating a non-malignant CNS disorder, the method comprising infusing a gene or RNA therapy vector into the pons of a subject in need thereof via (CED).

25. The method of claim 24, wherein the gene or RNA therapy vector is infused into the anterior pons.

26. The method of claim 24, wherein the gene or RNA therapy vector is also infused into the middle cerebellar peduncle.

27. The method of claim 24, wherein the infusion is through at least one cannula placed via a trans-cerebellar posterior to anterior trajectory traversing laterally to the deep cerebellar nuclei.

28. The method of claim 24, wherein the cannula is a stepped cannula.

29. The method of claim 28, wherein the cannula step is placed at the interface of the posterior deep cerebellar nuclei and cerebellar white matter.

30. The method of claim 24, wherein the gene or RNA therapy vector is infused into the pons to deliver the gene or RNA therapy vector to one or more of the ascending and descending sensory and motor pathways, brainstem nuclei, the deep cerebellar nuclei, cerebellar white matter, cerebellar Purkinje cells and the inferior olivary nucleus by anterograde and/or retrograde transport.

31. The method of claim 24, wherein the gene or RNA therapy vector is a viral or a non-viral vector.

32. The method of claim 31, wherein the gene or RNA therapy vector is a viral vector selected from adenoviruses, adeno-associated viruses (AAVs), lentiviruses, and herpes viruses.

33. The method of claim 32, wherein the gene or RNA therapy vector is AAV5 or AAV9.

34. The method of claim 32, wherein the viral vector is administered at a concentration of from 10⁵ to 10¹⁵ Vg/ml.

35. The method of claim 31, wherein the gene or RNA therapy vector is a non-viral vector selected from polymers, lipids, peptides, inorganic materials and hybrid systems.

36. The method of claim 24, wherein the non-malignant CNS disorder is selected from neurodegenerative diseases, neurological diseases, enzyme deficient conditions, neuroinflammatory diseases, acquired neurological injuries, or neurological disorders.

37. The method of claim 36, wherein the neurological disease is:
a) a disease characterized by degeneration of predominantly pyramidal motor pathways;
b) a disease characterized by degeneration of predominantly extrapyramidal motor pathways;
c) a disease characterized by degeneration of predominantly cerebral cortex: or
d) a disease characterized by predominant degeneration of the cerebellar system.

38-40. (canceled)

41. The method of claim 36, wherein the neurological disease is a disease characterized by disorders of the limbic system.

42. The method of claim 36, wherein the neurological disease is a disease characterized by widespread pathological changes throughout the nervous system.

43. The method of claim 36, wherein the neurological disease is an enzyme deficiency disorder.

44. The method of claim 36, wherein the neurological disease is a neuroinflammatory disorder.

45. The method of claim 36, wherein the neurological disease is a neuropsychiatric disorder.

46. The method of claim 36, wherein the acquired neurological injury is stroke, traumatic brain injury or spinal cord injury.

47. A method of distributing a gene therapy throughout the nervous system, wherein a viral vector that is axonally transported anterogradely and retrogradely is infused into the pons and middle cerebellar peduncle of a subject via a cannula that is inserted through the cerebellum, the middle cerebellar peduncle and into the pons.

48-75. (canceled)
